SEQUENCE LISTING (7 sequences)

SEQ ID NO: 1 - protein, 10 aa, Artificial Sequence
  Description of Artificial Sequence Synthetic peptide
NVYGIESVKI

SEQ ID NO: 2 - protein, 10 aa, Artificial Sequence
  Description of Artificial Sequence Synthetic peptide
NVIYVKSEIG

SEQ ID NO: 3 - protein, 10 aa, Artificial Sequence
  Description of Artificial Sequence Synthetic peptide
QMNQIQSVEV

SEQ ID NO: 4 - protein, 10 aa, Artificial Sequence
  Description of Artificial Sequence Synthetic peptide
LHLHGTGIQV

SEQ ID NO: 5 - protein, 7 aa, Artificial Sequence
  Description of Artificial Sequence Synthetic peptide
DIEGRMD

SEQ ID NO: 6 - protein, 6 aa, Artificial Sequence
  Description of Artificial Sequence Synthetic peptide
IEGRMD

SEQ ID NO: 7 - protein, 5 aa, Artificial Sequence
  Description of Artificial Sequence Synthetic His tag sequence
HHHHH

(19) United States
(12) Patent Application Publication
(10) Pub. No.: US 2004/0241754 A1
(43) Pub. Date: Dec. 2, 2004

(54) Modulating neuronal plasticity

(75) Inventor: Stephen F. Heinemann, La Jolla, CA (US)

Correspondence Address: FOLEY & LARDNER, P.O. BOX 80278, SAN DIEGO, CA, 92138-0278, US

(73) Assignee: The Salk Institute for Biological Studies

(21) Appl. No.: 10/449,715

(22) Filed: May 31, 2003

(51) Int. Cl. G01N 33/53; G01N 33/567

(52) U.S. Cl. 435/7.2

(57) ABSTRACT

Methods of identifying compounds that modulate neuronal plasticity including long-term potentiation are identified by their ability to modulate association between an Eph receptor and an ephrin ligand, or between an Eph receptor and a PDZ protein. In another embodiment the method for screening compounds involves applying the candidate compounds to a postsynaptic neuron and determining a biochemical affect on the presynaptic neuron, or by determining clustering of Eph receptors or ephrin ligands. Also provided are methods for using the invention compounds to modulate plasticity in an individual in need thereof or to improve cognition in an individual.

### STATEMENT AS TO FEDERALLY-FUNDED RESEARCH

This invention was supported in part by government funding from the National Institutes of Health under grant no. 5 R01 NS28709-06_{—}10. The United States government may have certain rights in this invention.

### BACKGROUND OF THE INVENTION

The present invention relates generally to the field of neuronal plasticity and to the identification of compounds and their use for modulating neuronal activity.

Synaptic plasticity, which is the ability of neural circuits to undergo changes in function or organization due to previous activity, is believed to play a significant role in aspects of cognition such as memory and learning. A simple form of neuronal plasticity known as “neural facilitation” is characterized by an increase in amplitude of a postsynaptic potential due to rapid repeated activation. The postsynaptic response is fleeting and the “facilitated” neuron returns to its resting potential between activations. In contrast, “neural potentiation” is a special type of facilitation in which an increased postsynaptic potential persists after the facilitating stimulus has subsided. For example, a high frequency burst of presynaptic impulses lasting several seconds, called a tetanic stimulus, can cause a posttetanic potentiation, (“PTP”) lasting only several minutes (i.e., short term potentiation). Extended stimulation, such as by tetanization, results in what is called long-term potentiation (“LTP”), the result of which is elevated postsynaptic activity for many minutes, hours or even days.

LTP is generally believed to play an important role in synaptic plasticity in mammalian CNS neurons, a process essential to memory and learning. The ability to control LTP, therefore, provides a therapeutic strategy for increasing cognition capability in a developing individual or in an individual with diminished cognition capacity. In the latter case, diminished capacity may be the result of any one of a variety of causes, e.g., disruption of neural network or death or dysfunction of constituent nerve cells achieved by neurodegenerative diseases and disorders, aging, trauma, exposure to harmful chemical or environmental agents.

### SUMMARY OF THE INVENTION

Accordingly, it is a general object of the present invention to provide methods and associated compositions for effectively modifying mammalian neurons to achieve a variety of beneficial results. In particular, the present invention is directed to modulating neuronal plasticity by interfering or affecting an ephrin receptor, ephrin ligand system located at synaptic junctions within CNS neurons. This system can be affected, for example, by interfering with the association of Eph receptors with their cognate ephrin ligands, altering clustering of Eph receptors or ephrin ligands, altering the association of Eph receptors or ephrin ligands and PDZ proteins or PDZ protein domains and other downstream postsynaptic or presynaptic events.

In these embodiments, the candidate compounds may be applied to a postsynaptic neuron and the biochemical effects detected at the presynaptic neuron. In further embodiments, the compounds are applied to the postsynaptic neuron and clustering of Eph receptors is determined in the postsynaptic neuron or clustering of ephrin ligands is determined in the presynaptic neuron.

In some embodiments, an EphB receptor or an ephrin ephrin-B ligand is involved. The neurons in these various embodiments may be located in the hippocampus, cerebellum, cortico-thalamic or amygdala. More specifically, the neurons are hippocampal mossy fiber CA3 neurons.

The invention compounds selected as described above are useful for modulating neuronal plasticity including long term potentiation of neurons.

Also provided are methods of modulating neuronal plasticity of an individual in need thereof by modulating the interaction between Eph receptors on postsynaptic neurons and ephrin ligands on presynaptic neurons. In an embodiment, modulation is achieved by contacting neurons of the individual with an invention compound.

Further provided are methods of modulating neural plasticity, e.g., improving cognition, in an individual by increasing clustering of Eph receptors at the synaptic site of postsynaptic neurons or by increasing clustering of ephrin ligands at the synaptic site of presynaptic neurons. In an embodiment, modulation is achieved by contacting neurons of the individual with an invention compound.

In some embodiments, an EphB receptor or an ephrin ephrin-B ligand is involved. The neurons in these various embodiments may be located in the hippocampus, cerebellum, cortico-thalamic or amygdala. More specifically, the neurons are hippocampal mossy fiber CA3 neurons.

Individuals treated in some embodiments may be suffering from a mental illness. In other embodiments, the mental illness is a defect in cognition.

These and additional embodiments of the invention are discussed in greater detail below.

### BRIEF DESCRIPTION OF THE DRAWINGS

FIG. 1 shows control tracings (no inhibitory peptide) and analysis demonstrating LTP induction in mossy fiber synapses of hippocampal neurons by the patch clamp technique. Time −30 min to 0 min represents baseline excitatory postsynaptic current (“EPSC;” 40 ms interval) while recordings after time zero represent post tetanic stimulation of EPSC. Panel in upper right shows a sample EPSC tracing taken pretetanic (“control”) and 20-30 minutes post tetanic (“LTP”). The left hand panel is a time course representing 13 experiments where mossy fiber LTP was evaluated in the absence of an inhibitory peptide. Potentiation of the EPSC is observed 20-30 minutes after tetanus. Application of the group II mGluR agonist, LCCG-1 [i.e., (2S,1′S,2′S)-2-(carboxycyclopropyl)glycine] at the end of each experiment (shown by thick bar) caused inhibition of the EPSC, signifying that the observed EPSCs are of mossy fiber origin. Panel in lower right shows mean paired pulse ratio of EPSCs prior to LTP (labeled “control”) and at 25 min post induction of LTP (labeled “LTP”). A reduced pulse paired result ratio (“PPR”; ratio of ESPC peaks) characteristic of LTP is observed.

FIG. 2 is otherwise similar to FIG. 1 in experimental design except that GluR2 carboxy terminal peptide (“GluR2ct”; SEQ ID NO:1) was included in the patch electrode to evaluate the affect of the peptide on LTP. Left panel shows results from 18 experiments indicating that little potentiation was observed between 25-30 minutes after tetanus in the presence of the GluR2ct peptide. Right upper panel shows sample traces from one recording. Calibration (for A & B): x axis, 50 ms; y axis 500 pA. Lower right panel with mean paired pulse ratios shows no significant difference after induction of LTP when the GluR2ct peptide is applied (p>0.05).

FIG. 3 is a cumulative probability histogram of hippocampal neuron mossy fiber LTP measured as % increase from control 25-30 minutes after tetanic stimulation. Left panel shows cumulative probability for the data shown in FIGS. 1 and 2. Left panel recordings in which the GluR2ct peptide was introduced into the postsynaptic cell (open circles) showed significantly less potentiation than recordings taken without peptide (filled squares) (p<0.01). Right panel shows cumulative probability when a scrambled version of the GluR2ct (“R2ctSC”; SEQ ID NO:2) or a phosphorylated version of the GluR2ct peptide (“R2ctPO4”) were added to the patch electrode. The result in both cases showed little effect on induction of LTP.

FIG. 4 is otherwise similar to FIG. 1 in experimental design except that an EphB2 carboxy terminal (“EphB2”; SEQ ID NO:3) peptide or an EphA7 carboxy terminal peptide (“EphA7”; SEQ ID NO:4) was added to the patch electrode. Left panel is a time course of mossy fiber LTP with inclusion of EphB2 peptide (open circles, n=7) or EphA7 carboxy terminal peptide (filled squares, n=5). LTP and PTP are significantly impaired in recordings with EphB2 peptide. Right hand panel shows sample EPSC traces from one recording before and after induction of LTP with EphB2 peptide (top) or EphA7 peptide (bottom). Calibration: x axis, 50 ms; y axis, 100 pA (EphB2), 200 pA (EphA7).

FIG. 5 is a cumulative probability histogram of % change in EPSC for hippocampal mossy fiber neurons 20-30 minutes after induction of LTP with EphB2 (SEQ ID NO:3) and EphA7 (SEQ ID NO:4) peptides.

FIG. 6 shows that a paired pulse ratio for hippocampal mossy fiber neurons after LTP induction is significantly reduced relative to pretetanic recording with EphA7 peptide (SEQ ID NO:4) as compared to recordings taken with EphB2 peptide.

FIG. 7 shows competitive binding inhibition curves for binding of labeled EphB2 c-term peptide (SEQ ID NO:3) to GRIP PDZ domains. Each data point represents the normalized mean of 4 experiments. Data points for the EphB2 peptide (open circles) and the GluR2ct peptide (filled squares) are fitted to the Hill equation. Top panel shows inhibition curves for GRIP PDZ4-6 which competed with increasing concentrations of peptide. The EphB2 peptide inhibits binding with a Ki of 43 nM and the GluR2ct peptide (SEQ ID NO:1) inhibits binding with a Ki of 106 nM. Labeled EphB2 was not displaced from GRIP by a scrambled version of the EphB2 carboxy terminus (“EphB2SC;” filled circles; SEQ ID NO:32) or by a phosphorylated version of EphB2 (“EphB2PO4;” filled triangles) at concentrations of up to 10 μM peptide. The bottom panel shows that GluR2ct can displace EphB2 from a GRIP fragment containing only the PDZ 6 domain.

FIG. 8 is a pull-down experiment in rat brain membranes using fusion proteins containing GST alone (lane 1) NMDA receptorla (NR1a) carboxy terminus fused to GST (lane 2) or EphB2 carboxy terminus fused to GST (lane 3). The results show specific association of GluR2 in brain membranes with EphB2.

FIG. 9 shows that antibody against the carboxy terminal portion of EphB2 receptor inhibits LTP for mossy fiber synapses of hippocampal neurons. Left panel is a time course of mossy fiber LTP conducted when either 20 μg/ml of an antibody against EphB2 (open circles, 12 recordings) or a sample of this antibody following preabsorption with EphB2 (filled squares, 8 recordings) are included in the patch electrode. A significant reduction in potentiation of the EPSC is observed in the presence of the antibody. In contrast, potentiation is restored when the antibody preparation is preabsorbed with the specific antigen (EphB2). Right panels show sample traces before and after LTP induction from individual recordings with EphB2 antibody (top) or pre-absorbed antibody (bottom). Calibration: x axis, 50 ms; y axis, 400 pA (EphB2 antibody), 600 pA (pre-absorbed antibody).

FIG. 10 shows that antibody against the amino terminal portion of the EphB1 receptor (“EphB1nt”) or to the carboxy terminal portion of the GluR2/3 (“GluR2/3ct”) receptor does not inhibit LTP of mossy fiber synapses. Left panel is a time course of LTP in recordings in which EphB1nt antibody (open circles) or GluR2/3ct antibody (filled squares) were included in the patch electrode. Right panel shows sample traces from recordings with EphB1nt antibody (top) and GluR2/3ct antibody (bottom). Calibration: x axis, 50 ms; y axis, 375 pA (EphB1 antibody), 500 pA (GluR2/3 antibody).

FIG. 11 is a cumulative probability histogram of LTP with inclusion of antibodies for the data shown in FIG. 10.

FIG. 12 shows that mean paired pulse ratio (PPR) after LTP is significantly reduced compared to pretetanic PPR in each case except when EphB2 antibody is present in the patch electrode. Paired pulse ratio values for each antibody are: EphB2a/b: control 2.8±1.9, LTP 2.9±0.2; pre-EphB2a/b: control 3.1±0.2, LTP 2.4±0.2; EphB1a/b: control 3.1±0.1, LTP 2.7±0.1; GluR2/3a/b: control 3.1±0.2, LTP 2.4±0.2.

FIG. 13 shows that application of soluble ectodomains of Eph receptors and ephrins inhibits hippocampal mossy fiber LTP. Upper left hand panel shows that extracellular application of EphB2-Fc (heavy bar) significantly increases basal synaptic transmission. Lower left panel shows that prior application of EphB2-Fc inhibits subsequent tetanus induced LTP. Lower left panel also shows that application of EphB2-Fc (heavy bar) does not effect basal synaptic transmission. Right upper panel shows sample traces before and during application of EphB2-Fc and after LTP induction (top). Lower right panel shows that mean paired pulse ratios were not significantly reduced after LTP induction in the presence of EphB2-Fc.

FIG. 14 left panel shows that application of ephrin-B1-Fc (heavy bar) does not affect basal transmission but does significantly impair LTP induction. Right upper panel are sample EPSC traces without peptide inhibitor before (“control”) and after tetanic stimulation (“LTP”) and in the presence of ephrin-B1-Fc. Lower right panel shows mean paired pulse ratios showing that bath inclusion of ephrin-B1-Fc showed no significant difference in PPR before or after induction of LTP.

FIG. 15 is a cumulative probability histogram of LTP induced during bath application of EphB2-Fc, ephrin-B1-Fc and EphA5-Fc soluble ectodomains. Significant potentiation was seen in recordings in the presence of EphA5-Fc but not with that of EphB2-Fc or ephrin-B1-Fc (EphA5-Fc control: 2.5±0.2; EphA5-Fc LTP: 1.9±0.1, n=6, p<0.05).

FIG. 16 upper graph shows that bath application of forskolin mediates enhancement of hippocampal mossy fiber transmission. Lower graph shows that application of ephrin-B1-Fe prior to forskolin (heavy bar) does not affect Forskolin enhancement while prior application of Eph B2-Fc inhibits subsequent forskolin effects.

FIG. 17 shows cumulative probability for forskolin enhancement of mossy fiber transmission with prior application of Fc fusion proteins or inclusion of EphB2 peptide in the recording electrode.

### DETAILED DESCRIPTION OF THE PREFERRED EMBODIMENTS

In accordance with the present invention, it has been discovered that the induction of LTP involves postsynaptic biochemical events, namely retrograde signaling through the Eph receptors/Eph ligands. Thus, induction of LTP initiated by activation of glutamate receptors (e.g. kainate and/or metabotropic receptors) upon tetanic stimulation of synaptic terminals and subsequent intracellular signaling results in the promotion of clustering of Eph receptors by GRIP or similar PDZ domain containing molecule. This interaction allows Eph receptors to associate with and activate reverse signaling via presynaptic ephrin ligands, which may then activate downstream events including clustering of the ephrin ligands and activation of PKA, ultimately increasing glutamate release from presynaptic terminals. The present invention therefore takes advantage of the discovery that LTP induction involves retrograde signaling from the post synaptic membrane to the presynaptic terminal through Eph receptor/ephrin ligand interactions, the end result being a long-lasting alteration in synaptic strength expressed presynaptically as an increased probability of neurotransmitter release.

Accordingly, the present invention provides a method of identifying a compound that modulates neuronal plasticity, said method comprising identifying those candidate compounds which modulate association between an Eph receptor or fragment thereof and an ephrin ligand or fragment thereof. In another embodiment, the invention provides methods of identifying compounds that modulate neuronal plasticity, said method comprising identifying those candidate compounds that modulate association between a PDZ protein or PDZ domain containing fragment thereof and an Eph receptor or fragment thereof. In yet a further embodiment, the invention provides methods of screening compounds as potential modulators of neuronal plasticity, said method comprising applying the compounds to postsynaptic neurons of a neuronal synapse and determining whether a biochemical affect is observed at the presynaptic neuron.

The term “neuronal plasticity” as used herein is the ability of neural circuits to undergo changes in function or organization due to previous activity. A simple form of neuronal plasticity known as “neural facilitation” is characterized by an increase in amplitude of a postsynaptic potential due to rapid repeated activation. The postsynaptic response is fleeting and the “facilitated” neuron returns to its resting potential between activations. In contrast, “neural potentiation” is a special type of facilitation in which an increased postsynaptic potential persists after the facilitating stimulus has subsided. For example, a high frequency burst of presynaptic impulses lasting several seconds, called a tetanic.

“Neuronal LTP” as used herein refers to neuronal potentiation characterized by a sustained increase in amplitude of a postsynaptic potential due to a facilitating stimulation, such as tetanization. Under LTP, the increase in postsynaptic potential persists well after the facilitating stimulus has subsided. In general, for cultured neurons or neural slices, short term potentiation (STP) is characterized by an increased potential that can be measured within 5 minutes post induction and substantially decays thereafter. LTP, in contrast, is characterized by an increased potential that can be measured within about 20-30 minutes post induction and substantially decays thereafter. For example, in the case of LTP for hippocampal CA3 neurons following tetanic stimulation as described in the examples, LTP is measured optimally between about 20 and 30 minutes post induction, more preferably between about 25-30 minutes post induction.

Plasticity involving LTP can be measured at any of a variety of CNS neuronal synaptic sites involved in this process. These include the hippocampal mossy fiber-CA3 synapses, cortico-thalamic synapes (Castro-Alamanfcos et al., J. Neurosci 19:9090-9097 (1999), cerebellar purkinje cell synapse (Salin et al., Neuron 16:797-803 (1996)), amygdala nerve synapses (Huang and Kandell, Neuron 21, 169-178 (1998)), and the like. Hippocampal mossy fiber CA3 neurons are a presently preferred source for inducing LTP and identifying modulating compounds.

The term “Eph receptor” as used herein refers to a family of receptors that contain an N-terminal Ig-like domain, a cysteine-rich region with 19 conserved cysteines, two fibronectin type III domains and a cytoplasmic region which contains a typical tyrosine kinase organization. Orioli et al., (1997) Trends Genet. 13:354; Zisch et al., (1997) Cell Tissue Res. 290:217. Eph receptors have been divided into two groups based on structural characteristics (e.g., the identity of extracellular domains) and the ability to bind preferentially to the ephrin-A or ephrin-B “ligand” proteins. See, e.g., Flanagan et al. (1997) Cell 90:403. Thus the Eph receptor family includes the called EphA receptors, characterized by interaction preferentially with ephrin-A ligands, and the EphB receptors, characterized by interaction preferentially with ephrin-B ligands.

As used herein, the term Eph receptor or EphR includes the full length receptor as well as fragments of the receptor that retain the ability to modulate LTP. Eph receptor fragments that can modulate LTP vary with assay in which they are used. For example the N-terminal domain of an Eph receptor can be an LTP inhibitor when applied extracellularly. In contrast, the C-terminal domain of the Eph receptor can be an LTP modulator if applied cytoplasmically to the postsynaptic neuron. Such fragments include the carboxy terminal portion of EphR, in particular, the terminal 10 amino acids of the receptor. Eph receptor fragments also can include less than a full length protein domain provided that biological activity is preserved. Fragments of an EphR characterized in having the ability to modulate LTP can be readily identified using the screening methods disclosed herein.

The EphA receptors include various distinct members designated EphA1 to EphA9. EphA1 binds to ephrin-A1, EphA2 through EphA8 bind to ephrins-A1 through -A5, and EphA4 binds to ephrin-B2 and B3. The EphB receptors include those designated EphB1 to EphB6. Eph receptors are defined as set forth by the Eph Nomenclature Committee, 1997. Cell, 90:404-404 (1997).

EphA1 (a.k.a. Eph, Esk): a 984 amino acid (amino acid) type I transmembrane protein with a predicted MW of 109 kDa. Hairi, et al. (1987) Science 238:1717. The molecule has a 23 amino acid signal sequence, a 524 amino acid extracellular region, a 21 amino acid transmembrane segment and a 416 amino acid cytoplasmic domain. Id. A partial mouse clone has been isolated and found to be approximately 80% identical to the human protein. Lickliter, et al. (1996) Proc. Natl. Acad. Sci. USA 93:145.

EphA2 (a.k.a. Eck, Myk2, Sek2): first isolated from keratinocytes, the molecule is 130 kDa and 976 amino acid long and contains a 17 amino acid signal sequence, a 517 amino acid extracellular segment, a 24 amino acid transmembrane region and a 418 amino acid cytoplasmic domain. EphA2 has also been found in Schwann cells, the primitive streak and hindbrain in a very restricted expression pattern. Ruiz, et al. (1994) Mech. Dev. 46:87.

EphA3 (a.k.a. Hek, Mek4, Cek4,Tyro4, Hek4): a 135 kDa, 983 amino acid type I transmembrane glycoprotein that contains a 20 amino acid signal sequence, a 521 amino acid extracellular region, a 24 amino acid transmembrane domain and a 418 amino acid cytoplasmic segment. Wicks, et al. (1992) Proc. Natl. Acad. Sci. USA 89:1611. The extracellular region has five N-linked glycosylation sites. The extracellular region of mouse and human EphA3 are 96% identical at the amino acid level. Sajjadi, et al. (1991) New Biologist 3:769. The mouse molecule may generate an alternatively spliced soluble form. Id.

EphA4 (a.k.a. Sek, Sek1, Ced8, Hek8, Tyrol): a 130 kDa, 963 amino acid transmembrane glycoprotein that contains a 528 amino acid extracellular region, a 22 amino acid transmembrane domain and a 417 amino acid cytoplasmic segment. Ellis, et al. (1996) Oncogene 12:1727; Fox, et al. (1995) Oncogene 10:897. Although the mouse and human extracellular regions are 98% identical at the amino acid level, there is a 24 amino acid addition in the human region. Fox, et al. (1995) Oncogene 10:897; Gilardi-hebenstreit, et al. (1992) Oncogene 7:2499. Cells that express EphA4 include keratinocytes, B cells and T cells. Ellis, et al. (1996) Oncogene 12:1727.

EphA5 (a.k.a. Bsk, Hek7, Ehk1,Cek7, Rek7): a 1037 amino acid transmembrane protein that is alternatively known as bsk for brain-specific kinase. Fox, et al. (1995) Oncogene 10:897;Zhou, et al. (1994) J. Neurosci. Res. 37:129. The protein consists of a 549 amino acid extracellular region, a 21 amino acid transmembrane segment and a 443 amino acid cytoplasmic domain. The mouse and human extracellular regions show 97% amino acid identity. Mouse EphA5 differs markedly from the human sequence in that it lacks a 164 amino acid insert. Thus, it contains only 12 cysteine residues and one fibronectin type III domain. Zhou, et al. (1994) J. Neurosci. Res. 37:129. In humans, there is a cytoplasmic alternate splice variant that contains a deletion of the kinase region. SWISS-PROT: Accession # P54756. The expression of EphA5 appears to be restricted to the brain. Fox, et al. (1995) Oncogene 10:897.

EphA6 (a.k.a. Ehk2,Hek12): identified in the mouse and is a 1035 amino acid transmembrane protein that consists a 22 amino acid signal sequence, a 521 amino acid extracellular region, a 25 amino acid transmembrane segment and a 467 amino acid cytoplasmic domain. Lee, et al. (1996) DNA Cell Biol. 15:817. Mouse and rat EphA6 are virtually identical at the amino acid level with the exception of 87 amino acid (a C-terminal extension in the mouse molecule). EphA6 is expressed in both adult and fetal cochlear ganglion cells. Id.

EphA7 (a.k.a. Hek 11, MCK1, Ehk3, Ebk, Cek11): a 998 amino acid type I transmembrane protein that contains a 24 amino acid signal sequence, a 532 amino acid extracellular region, a 21 amino acid transmembrane domain and a 421 amino acid cytoplasmic segment. It has been found on fetal pro- and pre-B cells.

EphA8 (a.k.a. Eek): a partial clone of human EphA8 has been reported. Chan, J. & V. M. Watt (1991) Oncogene 6:1057. The mouse receptor is a 120 kDa, 977 amino acid type I transmembrane glycoprotein with a 513 amino acid extracellular region, a 21 amino acid transmembrane domain and a 443 amino acid cytoplasmic segment. Id. EphA8 is considered specific for glycosyl phosphatidylinositol (“GPI”)-linked ligands and exhibits a Kd of 1.3 nM for ephrin-A2 binding, a Kd of 1.1 nM for ephrin-A3 binding, and a Kd of 500 pM for ephrin-A5 binding.

EphB1 (a.k.a. Elk, Net, Cek6, Hek6): a 967 amino acid transmembrane protein that contains a 523 amino acid extracellular region, a 20 amino acid transmembrane domain and a 424 amino acid cytoplasmic segment. Rat and human EphB1 are 99% identical at the amino acid level. Tang, et al. (1995) Genomics 29:426. EphB1 is found on endothelial cells and is activated by ephrin-B1, an event that initiates the assembly of endothelial cells into capillary-like cords. Stein, et al. (1996) J. Biol. Chem. 271:23588; Daniel, et al. (1996) Kidney Int. (Suppl) 57:S73.

EphB2 (a.k.a. Erk and Nuk): a 969 amino acid, type I transmembrane protein that contains a 522 amino acid extracellular region, a 26 amino acid transmembrane segment and a 421 amino acid cytoplasmic domain. Ikegaki, et al. (1995) Human Mol. Genet. 4:2033. Mouse and human EphB2 are 99% identical at the amino acid level. Henkemeyer, et al. (1994) Oncogene 9:1001. EphB2 seems to be transiently expressed on axons only during their outgrowth or migration.

EphB3 (a.k.a. Hek2 and MDK5): a 130 kDa, 998 amino acid transmembrane glycoprotein that contains a 33 amino acid signal sequence, a 523 amino acid extracellular region, a 26 amino acid transmembrane domain and a 416 amino acid cytoplasmic segment. Bohme, B. et al. (1993) Oncogene 8:2857. In the adult, it is apparently expressed on macrophages. The extracellular regions of mouse and human EphB3 are 96% identical at the amino acid level. Ciossek, et al. (1995) Oncogene 11:2085.

EphB4 (a.k.a. Htk and MDK2): a 120 kDa, 972 amino acid type I transmembrane glycoprotein with a 524 amino acid extracellular region, a 21 amino acid transmembrane segment and a 427 amino acid cytoplasmic domain. Bennett, et al. (1994) J. Biol. Chem. 269:14211. The extracellular regions of mouse and human are somewhat varied, showing only 88% amino acid identity. Ciossek, T. et al. (1995) Oncogene 11:2085; Bennett, et al. (1994) J. Biol. Chem. 269:14211; Andres, et al. (1994) Oncogene 9:1461. EphB4 is found on CD34+stem cells, (Id.) BFU-E26 and secretory mammary epithelium. Berclaz, et al. (1996) Biochem. Biophys. Res. Commun. 226:869.

EphB5 (a.k.a. Cek9): reported in the chicken as 1000 amino acid long molecule with a 29 amino acid signal sequence, 529 amino acid extracellular domain, 24 amino acid transmembrane region and 418 amino acid cytoplasmic segment. Consistent with other EphR, the extracellular region has 19 conserved cysteines and two fibronectin type III domains. Soans, et al. (1996) J. Cell Biol. 135:781.

EphB6 (a.k.a. Hep and Mep): a 135 kDa type I transmembrane glycoprotein that contains of a 561 amino acid extracellular region, a 26 amino acid transmembrane segment and a 403 amino acid cytoplasmic domain. Matsuoka, et al. (1997) Biochem. Biophys. Res. Commun. 235:487. There is 93% amino acid sequence identity between mouse and human EphB6. Gurniak, et al. (1996) Oncogene 13:777. In both the human and mouse, the kinase domain is inactive. The function of such a receptor is unknown. The non-functionality of the receptor is further complicated by the fact that, in the mouse, there is a possibility of an alternatively spliced secreted form. Id.

“Ephrin ligand” as used herein refers collectively to a family of membrane proteins that act as ligands for the Eph family of receptors. Ephrin ligands include ephrin-A subclass ligands, which are glycosylphosphatidylinositol (GPI)-linked membrane proteins, and the ephrin-B subclass ligands, which are transmembrane linked membrane proteins. These two subgroups of ephrin ligands are distinguished structurally on the basis of their amino acid sequence and functionally on the basis of their preferential binding to two corresponding receptor subgroups; the ephrin A subclass ligands bind to the EphA receptors and ephrin-B subclass ligands bind to the EphB receptors. The ephrin-A ligands include those designated ephrin-A1 to ephrin-A6 while the ephrin-B ligands include those designated ephrin-B1 to ephrin-B3. Ephrin ligands are defined as set forth by the Eph Nomenclature Committee, 1997, 1997. Cell, 90:404-404 (1997).

The B type (transmembrane) ephrin ligand can transduce a signal upon binding to an appropriate Eph receptor. Holland, et al. (1996) Nature 383:722. Apparently, ephrins need to be membrane-bound to activate Ephs, as soluble forms of class A and B ephrins are inactive in Eph phosphorylation assays. Davis, et al. (1994) Science 266:816. Ephrins demonstrate four conserved cysteines in their mature segments. Overall, class A ephrins show 23% amino acid (amino acid) identity in their mature regions, (Kozlosky, et al. (1997) Cytokine 9:540; Cerretti, et al. (1998) Genomics 47:131) while class B ephrins share 33% amino acid identity in their extracellular segments and 44% amino acid identity in their cytoplasmic regions. Nicola, et al. (1996) Growth Factors 13:141. In general, class A ephrins bind to class A Eph receptors, while class B ephrins bind to class B Eph receptors. Gale, et al. (1997) Cell Tissue Res. 290:227; Orioli, et al. (1997) Trends Genet. 13:354.

As used herein, the term ephrin ligand includes the full length ephrin ligand as well as fragments of the ligand that retain the ability to modulate LTP. Ephrin ligand fragments that can modulate LTP vary with assay in which they are used. For example the N-terminal domain of an ephrin ligand can be an LTP inhibitor when applied extracellularly. The ephrin B2-Fc receptor fusion protein is an example of such an ephrin ligand fragment. In contrast, the C-terminal domain of the ephrin ligand can be an LTP modulator if applied cytoplasmically to the presynaptic neuron. The PDZ recognition motif in the C-terminal domain of an ephrin ligand is an example of an ephrin ligand fragment that modulates LTP. Ephrin ligand fragments also can include less than a full length protein domain provided that biological activity is preserved. Fragments of an ephrin ligand characterized in having the ability to modulate LTP can be readily identified using the screening methods disclosed herein.

Ephrin-A1 (a.k.a. B61 and LERK-1): a 25 kDa, 205 amino acid glycoprotein that has an 18 amino acid signal sequence and a 187 amino acid mature segment. The C-terminal 23 amino acid are believed to participate in GPI-linkage. Ephrin-A1 is inducible on endothelial cells by both TNF-a and IL-1b14 and can be found on fetal osteoblasts, odontoblasts, chondrocytes, and squamous epithelium. Takahashi, et al. (1995) Oncogene 11:879. The mature segments of mouse and human ephrin-A1 demonstrate 85% amino acid identity. Id. Ephrin-A1 binding to EphA2 has a Kd=25 nM. Bartley, et al. (1994) Nature 368:558.

Ephrin-A2 (a.k.a. ELF-1 and LERK-6): a 213 amino acid protein that contains a 20 amino acid signal sequence and a 193 amino acid mature segment. The mature segment has six cysteines and two potential N-linked glycosylation sites. Cerretti, et al. (1998) Genomics 47:131. Mouse and human ephrin-A2 share 90% amino acid identity in the mature segment. Id., Cheng, et al. (1994) Cell 79:157. EphA3 and EphA4 bind to ephrin-A2 with Kds of 1 nM and 10 nM, respectively. Id.

Ephrin-A3 (a.k.a. LERK-3): a 238 amino acid polypeptide that contains a 22 amino acid signal sequence and a 216 amino acid mature segment. The mature segment has six cysteines and three potential N-linked glycosylation sites. Kozlosky, et al. (1995) Oncogene 10:299. Ephrin-A3 binding to EphA3 has a Kd=5 nM. Id. Ephrin-A3 is noted for its expression in the olfactory system. Zhang, et al. (1996) J. Neurosci. 16:7182.

Ephrin-A4 (a.k.a. LERK-40): a 201 amino acid polypeptide with a 22 amino acid signal sequence and a 179 amino acid mature segment. Kozlosky, et al. (1995) Oncogene 10:299. The mature protein has one potential N-linked glycosylation site and seven cysteines. Ephrin-A4 binds to EphA4 with a Kd=5 nM and to EphB1 with a Kd 20 nM. Id. Mouse and human ephrin-A4 show 86% amino acid identity in the mature segment. Cerretti, et al. (1998) Genomics 47:131.

Ephrin-A5 (a.k.a. AL-1 and Lerk-7): a 28 kDa, 228 amino acid glycoprotein that contains a 20 amino acid signal sequence and a 208 amino acid mature segment. The mature segment contains six cysteines and one N-Linked glycosylation site. Kozlosky, et al. (1997) Cytokine 9:540.; Winslow, J. W. et al. (1995) Neuron 14:973. Between mouse and human ephrin-A5, there is 99% amino acid identity in the mature segment. Flenniken, et al. (1996) Dev. Biol. 179:382. In the mouse, there is also an alternatively spliced short form (a 27 amino acid deletion) which may be reflected in the human. Ephrin-A5 is found on astrocytes and skeletal muscle.

Ephrin-B1 (a.k.a. Elk-L and LERK2): a 45 kDa, 346 amino acid glycosylated polypeptide that contains a 24 amino acid signal sequence, a 211 amino acid extracellular region, a 26 amino acid transmembrane (transmembrane) domain and an 83 amino acid cytoplasmic segment. Davis, et al. (1994) Science 266:816; Beckman, et al. (1994) EMBO J. 13:3657. There is 95% amino acid identity in the extracellular segment of mouse and human ephrin-B1. Shao, et al. (1994) J. Biol. Chem. 269:26606. The Kd for ephrin-B1 binding to EphB1 is 925 pM, while the Kd for ephrin-B1 binding to EphA3 is 350 nM, emphasizing the general class specificity of the ephrins. Beckman, et al. (1994) EMBO J. 13:3657. A potential proteolytic cleavage site on ephrin B1 has been identified. Id.

Ephrin-B2 (a.k.a. Htk-L, LERK-5 and NLERK-1): a 38-42 kDa, 333 amino acid glycoprotein with a 25 amino acid signal sequence, a 199 amino acid extracellular region, a 26 amino acid transmembrane segment and an 83 amino acid cytoplasmic domain. Nicola, et al. (1996) Growth Factors 13:141; Cerretti, et al. (1995) Mol. Immunol. 32:1197. There is 98% amino acid identity in the extracellular region of mouse and human ephrin-B2. Id.; Bennett, et al. (1995) Proc. Natl. Acad. Sci. USA 92:1866. Ephrin-B2 is found on bone marrow fibroblasts, (Inada, et al. (1997) Blood 89:2757) activated melanocytes and melanoma cells, (Id.), monocytes, mesangial cells and CD34+stem cells (Bennett, et al. supra (1995). The Kd for ephrin-B2 binding to EphB4 is 535 pM. Id.

Ephrin-B3 (a.k.a. Elk-L3 and NLERK-2): a 50 kDa, 340 amino acid glycoprotein that contains a 28 amino acid signal sequence, a 196 amino acid extracellular region, a 25 amino acid transmembrane region and a 91 amino acid cytoplasmic domain. Nicola, et al. (1996) Growth Factors 13:141; Gale, et al. (1996) Oncogene 13:1343. Based on the extracellular region, there is evidence for proteolytic cleavage of this ligand. Nicola, N. A. et al. supra (1996). There is 95% amino acid identity in the extracellular regions of mouse and human ephrin-B3. Bergemann, et al. (1998) Oncogene 16:471. When the extracellular regions for all three B class ligands are aligned, pairwise comparisons demonstrate 50% amino acid identity for ephrin-B1 and ephrin-B2, 42% amino acid identity for B1 and B3, and 38% amino acid identity for ephrin-B2 and ephrin-B3. Id.

Postsynaptic Density disc-large ZO-1 protein or “PDZ protein” as used herein is an intracellular signaling protein associated with the plasma membrane and which mediates formation of membrane-bound macromolecular complexes of receptors and channels. PDZ proteins usually achieve complexing of receptors and channels by homotypic interaction. PDZ domain proteins usually bind to short linear C-terminal sequences in the protein with which they interact. Glutamate receptor interacting protein, or “GRIP”, is a 120 kD (1112 amino acid residues) PDZ protein present in postsynaptic terminals. GRIP contains 7 PDZ domains of which PDZ domains 4 and 5 are involved in the clustering AMPA receptors. The amino acid and cDNA for PDZ proteins are published and available in sequence repositories. For example, the amino acid and encoding DNA for human GRIP1 is published (Bruckner et al., (1999) Neuron 22 (3), 511-524) and the sequence is available in the NCBI (GenBank) under accession no. AJ133439.

A variety of other PDZ proteins are known in neural tissue and include, for example, Afadin (AF6) (Hock et al. Proc. Natl. Acad. Sci. USA, 18;95(17):9779-84 (1998); Nishioka et al. J. Comp. Neurol. 21;424(2):297-306 (2000)), CASK (Hsuch et al., J. Cell Biol. 13;142(1):139-51 (1988)), syntenin (Hirbec et al., J. Biol. Chem. 277(18):15221-4 (2000), PSD-ZIP45, and the like. Clustering of ephrin ligands during plasticity also may involve interaction with a PDZ protein, for example, PICK1 (Hirbec et al., supra).

The term PDZ protein as used herein also includes peptides with one or more PDZ domains. PDZ domain 6 of GRIP is an example of a PDZ protein as used herein. Fragments of PDZ full length proteins are well known in the art. See H. Dong et al., Nature 386, 279-84. (1997); see also examples.

As already discussed, one embodiment of the invention relates to identifying compounds that modulate neuronal LTP. This is accomplished in one approach by determining if a compound modulates association between an Eph receptor and its cognate ephrin ligand. The particular Eph receptor that is involved in LTP induction can vary with the neuronal cells. For example, an EphB receptor is involved in induction of LTP for mossy fiber synaptic junctions of hippocampal CA3 neurons, while an EphA receptor is not involved (see examples). One skilled in the art can readily determine which Eph receptors (or corresponding ephrin ligands) are involved in mediating LTP for a particular neuron using the methods disclosed herein.

A useful method for identifying if a candidate compound modulates LTP is the patch clamp technique. The patch clamp method is well known in the art (see e.g., Penner, (1994) A Practical Guide to Patch Clamping In “Single Channel Recording,” (B. Sackmann and E. Neher, Eds.), Chapter 1, Plenum, New York) and has been used to measure LTP. Briefly, the patch clamp technique allows measurement of ion flow through single ion channel proteins, and also allows the study of the single ion channel response to drugs. In general, in standard patch clamp technique, a thin (<1 micron in diameter) glass pipette is used. The tip of the pipette is pressed against the surface of the cell membrane. The pipette tip seals tightly to the cell and isolates a few ion channel proteins in a tiny patch of membrane. The activity of these channels can be measured electrically (single channel recording) or, alternatively, the patch clamp can be ruptured allowing measurements of the channel activity of the entire cell membrane (whole cell recording). During both single channel recording and whole-cell recording, the activity of individual channel subtypes can be further resolved by imposing a “voltage clamp” across the membrane. Through the use of a feedback loop, the “voltage clamp” imposes a voltage gradient across the membrane, limiting overall channel activity and allowing resolution of discrete channel subtypes.

A competitive binding assay can also be used to identify compounds that modulate LTP. Various in vitro assay formats well known in the art are useful for this purpose. For example, the assay can involve measuring binding of a labeled soluble member to its cognate partner attached to a solid phase. An Eph receptor or fragment thereof can be attached to a solid phase and then contacted with a soluble form of the appropriate ephrin ligand or fragment thereof (or vice versa). Association between these forms can then be evaluated in the presence of a candidate compound. Similarly, candidate compounds can be tested for their effect on binding between a PDZ domain protein or fragment thereof in soluble form and Eph receptor or fragment thereof on a solid phase (or vice versa). Various types of solid phases suitable for use in such assays are known including, organic or inorganic, or a combination of any of these—in the form of particles, strands, precipitates, gels, sheets, tubing, spheres, containers, capillaries, pads, slices, films, plates, slides, and the like. Typical supports are made of glass, plastic, or nylon. The examples herein describe a binding assay between GRIP PDZ domain fragments (e.g., His-GRIP-PDZ4-6) and an EphB2 carboxy terminal fusion protein labeled with ³⁵S-methionine (³⁵S-EphB2).

Proteins or peptides may be labeled with any of a variety of well known detectable agents such as radioisotopes (e.g., iodine, indium, sulfur, hydrogen etc.) a dye or fluorophor (e.g., cyanine, fluorescein, rhodamine), protein (e.g., avidin, antibody), enzyme (peroxidase, phosphatase, etc.), or any other agent that can be detected directly or indirectly. An enzyme is an example of a detectable moiety detected by indirect means. In this case, the enzyme is attached to a polypeptide and the presence of the enzyme is detected by adding an appropriate substrate that when acted upon by the enzyme, causes the substrate to change in color or to release a cleavage product that provides a different color from the original substrate.

A detectable moiety may include more than one chemical entity such as in fluorescent resonance energy transfer (“FRET”). In FRET based assays, interaction between biomolecules is measured indirectly by conjugating one of a pair of carefully selected fluorescent dyes to each of the molecules of interest. The absorption spectrum of the acceptor must overlap fluorescence emission spectrum of the donor and donor and acceptor transition dipole orientations must be approximately parallel. For instance, see Ju et. al. (1995) Proc. Natl. Acad. Sci. (USA) 92: 4347. However, in all cases, labeling should not interfere with binding of the cognate partners.

“Association” between an Eph receptor and its cognate ephrin ligand as used herein refers to the affinity or extent of interaction between these two molecules. The association or affinity between two molecules that interact can be evaluated by determining a binding constant or an association constant. Affinity is calculated as K_{d}=k_{off}/kₒₙ. The affinity can be determined at equlibrium by measuring the fraction bound (r) of labeled ligand at various concentrations (c). The data are graphed using the Scatchard equation: r/c=K(n−r):

r=moles bound ligand/mole receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. no. 6,316,409).

“Modulate association” as used herein means that the association constant or binding constant between two molecules is increased or decreased. For example, compounds that modulate association between an Eph receptor and an ephrin ligand may increase or decrease the association that naturally exits between these two molecules. Similarly, compounds that modulate association between an Eph receptor and a protein with a PDZ domain may increase or decrease the association that naturally exits between these two molecules. An increase or decrease in association can be measured by a change in the association constant, which may reflect a change in the on-rate or off-rate. Methods to measure the association constant or on- or off-rate are well known in the art (see also examples).

Association between molecules such as proteins can be determined using the full length protein or fragments such as polypeptides or peptides that retain sequence necessary for binding. As used herein “protein,” polypeptide,” and “peptide” are used interchangeably to refer to a polymer of amino acid residues linked by amide bonds. The terms apply to amino acid polymers in which one or more amino acid residue is an artificial chemical analogue of a corresponding naturally occurring amino acid, as well as to naturally occurring amino acid polymers.

Candidate compounds to test as LTP modulators can be obtained from a wide variety of sources including libraries of synthetic or natural compounds. For example, numerous means are available for random and directed synthesis of a wide variety of organic compounds and biomolecules, including expression of randomized oligonucleotides and oligopeptides. See, for example, U.S. Pat. No. 5,877,030 to Rebek et al. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, and the like, to produce structural analogs. Candidate compounds can be found among biomolecules including, but not limited to: peptides, saccharides, fatty acids, steroids, purines, pyrimidines, derivatives, structural analogs or combinations thereof.

The present invention also provides compounds that modulate neuronal LTP. Such compounds include peptides, peptidomimetics, polypeptides, pharmaceuticals, chemical compounds, biological agents, and the like. Antibodies, neurotropic agents, anti-epileptic compounds and combinatorial compound libraries can also be tested using the methods of the invention. One class of compound contemplated for modulating LTP is an organic molecule, preferably having a molecular weight of more than 50 and less than about 2,500 Daltons, more preferably less than about 1,000 Daltons and even more preferably less than about 700 Daltons. Invention compounds preferably are capable of crossing the blood brain barrier.

Compounds of the invention contain functional groups necessary for structural interaction with proteins, particularly interaction via hydrogen bonds, such compounds typically comprising at least an amine, carbonyl, hydroxyl or carboxyl group, and preferably at least two such functional groups. The compounds also may comprise carbocyclic or heterocyclic structures and/or aromatic or polyaromatic structures substituted with one or more of the above functional groups.

Specific examples of LTP modulating compounds provided herein include GluR2ct peptide (Asn-Val-Tyr-Gly-Ile-Glu-Ser-Val-Lys-Ile; SEQ ID NO:1) and the EphB2 peptide (Gln-Met-Asn-Gln-Ile-Gln-Ser-Val-Glu-Val; SEQ ID NO:3). One or ordinary skill realizes that modulators can include larger portions of the c-terminus of GluR2ct or an EphBct and the peptide may be used as part of a larger protein sequence. The above peptides inhibit mossy fiber hippocampal CA3 neuronal LTP if applied intracellularly to postsynaptic neurons. Multimer forms of the peptides in which the peptide sequence is repeated three or more times in a single molecule (e.g., trimer, tetramer, and the like) have the ability to cluster Eph receptors. Thus, such multimers can be used to increase LTP as opposed to monomer or dimer forms of the peptide.

Other modulators include PDZ proteins. In a preferred embodiment, the modulator is specific for type II PDZ domain, such as GRIP. In more preferred embodiments, the PDZ domains such as from GRIP including GRIP PDZ(4-6) (amino acids 415-801 of GRIP-1) and GRIP(6) (amino acids 634-912 of GRIP) are useful as modulators. Useful modulators may modulate binding between GRIP and EphR but not affect binding to PICK1 PDZ.

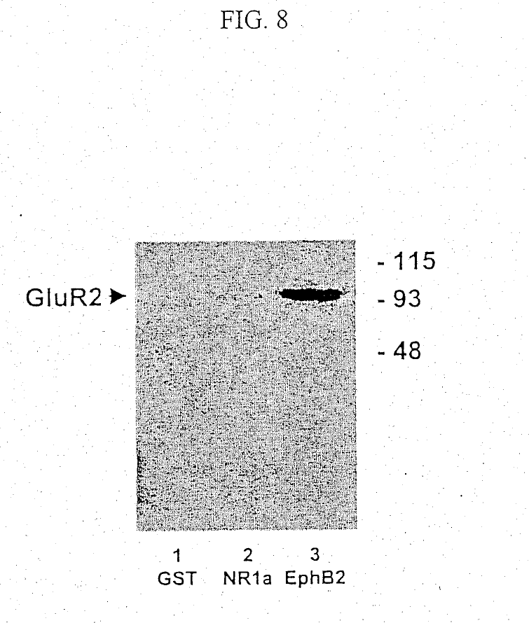

The inhibitory effect of the GluR2ct peptide is sequence specific and requires the presence of the carboxy terminal PDZ-binding region. Although not wishing to be bound by any theory, the GluR2ct peptide competitively inhibits binding between GRIP PDZ domain fragments and the EphB2 carboxy terminus (displacement having a Ki of 106 nM). Peptides GluR2ctSC (Asn-Val-Ile-Tyr-Val-Lys-Ser-Glu-Ile-Gly; SEQ ID NO:2) and GluR2ctPO₄ (same as SEQ ID NO:2 except that the serine residue is phorphorylated) that do not inhibit binding of EphB2 to GRIP at concentrations as high as 10 μM also do not inhibit LTP. As shown herein, the GluR2ct peptide displaces EphB2 binding directly to PDZ domain 6 (Ki value of 155 nM) rather than through an allosteric mechanism. GluR2 specifically associates with EphB2 but not with NMDA receptors (FIG. 8) indicating that EphB2 and GluR2 can potentially bind to the same GRIP complex in vivo. The GluR2 carboxy terminus, unlike the peptide derived therefrom, does not bind to PDZ domain 6 of GRIP in yeast (Dong et al., (1997) Nature 386, 279-84). Thus, the GluR2ct peptide, unlike the carboxy terminal domain of the GluR2 protein, binds directly to PDZ domain 6 of GRIP and disrupts the interaction between GRIP and Eph receptors. The EphB2 peptide may also interfere with binding of other proteins to GRIP PDZ domain 6 as shown by the fact that LTP is inhibited using antibodies against the carboxy terminus of EphB2 (EphB2a/b).

LTP Modulatory compounds provided herein also can modulate association between the EphRs and their intercellular binding partners, the ephrin ligands. EphR/ephrin intercellular signaling pathways are important for axon guidance and neuronal migration during development (R. Klein, Curr. Opin. Cell Biol. 13, 196-203. (2001)), but these molecules also are expressed at synapses in adult brain (R. Torres et al., Neuron 21, 1453-63. (1998); Buchert et al., (1999) J. Cell Biol. 144, 361-71), including in the CA3 region of the hippocampus (Moreno-Flores, et al. (1999) Neuroscience 91, 193-201), and they interact with the same PDZ proteins as AMPA receptors, albeit at different PDZ domains (Torres et al., (1998) Neuron 21, 1453-63). An example is the EphB2 receptor and peptides derived therefrom. As shown in the Examples, the EphB2 peptide (SEQ ID NO: 3) significantly depressed both post-tetanic potentiation and tetanus induced LTP.

Modulatory compounds also may be antibodies. Antibodies specific for carboxy-terminal domain of the Eph receptor are shown to modulate LTP. The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The terms variable light chain (VL) and variable heavy chain (VH) refer to these light and heavy chains, respectively. The N-terminus of each chain defines a variable region of about 100 to 110 or more amino acids primarily responsible for antigen recognition. An antibody can be specific for a particular antigen. The antibody or its antigen can be either an analyte or a binding partner.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the F(ab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

A single chain Fv (“scFv”) polypeptide is a covalently linked VH::VL heterodimer which may be expressed from a nucleic acid including VH- and VL-encoding sequences either joined directly or joined by a peptide-encoding linker. Huston, et al. (1988) Proc. Nat. Acad. Sci. USA, 85:5879-5883. A number of structures are known for converting the naturally assembled—but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs.” Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface.” This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987). An epitope is that portion of an antigen that interacts with an antibody.

Modulators also include chimeric protein reagents that comprise an Eph receptor or ephrin ligand extracellular domain, or a PDZ protein (or PDZ domain) fused to another protein. Such other protein may allow multimer formation such as when IgG Fc is used (generally forms a dimer). A segment of human IgG heavy chain from amino acid 100-330 can, for example, be used to produce such a dimer. IgM Fc can be used to form a pentamer if J chain also is included. A polypeptide sequence providing a linker also may be included between the two ends of the fusion protein. In addition, other functional peptide sequences such as a purification tag or labeling tag (e.g., poly His sequence) may be included in the chimeric protein. The EphB2 extracellular domain fused to IgGFc described herein is an example of such a chimeric protein. Methods of preparing chimeric proteins are well known in the art. In addition, chimeric proteins are available commercially (see, e.g., R&D Systems, Minneapolis, Minn.; Catalog nos. 467-B2 and 473 EB).

While application of pre-clustered Eph-Fc and ephrin-Fc reagents can activate reverse or forward signaling, respectively, the dimeric forms act primarily as blocking reagents to prevent signaling, and display only weak agonist activity. See, Stein et al., Genes Dev 12, 667-78. (1998). For example, dimeric EphB-Fc fusion proteins can partially activate presynaptic ephrin-B ligands and potentiate mossy fiber synaptic transmission, thus occluding subsequent tetanus induced LTP. Soluble ephrin-Fc ligands block signaling from postsynaptic EphB receptors and significantly impair LTP induction.

Although EphB2 receptors play a role in mossy fiber LTP, other Eph receptors are involved. This is based on recordings from EphB2 carboxy terminal truncation mutants which showed no obvious gross anatomical abnormalities in the CA3 region of EphB2 knockout mice and mossy fiber inputs were easily identifiable. Mossy fiber LTP in these mice was normal compared to recordings from heterozygous or wildtype littermates. Thus, other members of the EphB receptor family are expressed in the CA3 and are likely to compensate for the absence of EphB2 in the mutant mice. In addition, NMDA and non-NMDA receptor signaling can generate LTP via the disclosed EphR/EphL interaction.

Another embodiment of the invention is a method of screening compounds as potential modulators of neuronal plasticity, said method comprising applying the compounds to postsynaptic neurons of a neuronal synapse and determining clustering of Eph receptors. A further embodiment is a method of screening compounds as potential modulators of neuronal plasticity, said method comprising applying the compounds to postsynaptic neurons of a neuronal synapse and determining clustering of presynaptic ephrin ligands.

The term “clustering” as used herein with respect to Eph receptors or ephrin ligands means and increase in the density of these molecules located at their respective sides of the synaptic junction. The individual receptors or ligands may be detected by using antibodies labeled with a detectable moiety such as a fluorescent dye. Such antibodies preferably are monomeric forms of an antibody (e.g. a Fab fragment) to avoid receptor or ligand clustering resulting from the antibody.

The distribution of detected receptors or ligands detected by labeled antibody can be visualized by microscopy. An increase in clustering may be determined by comparing the fluorescence image before and after LTP induction. If clustering is observed, fluorescence is increased in localized areas at the synaptic junction. One can introduce candidate compounds into this assay to determine their impact on receptor or ligand clustering.

Clustering is preferably evaluated using a high resolution microscope. High resolution microscopes and are well known in the art as are devices for achieving high sample throughput (see e.g., WO0019262A2 to Kauvar et al.). Digital imaging also may be used to evaluate the fluorescent images and clustering may be determined using computer software. Commercial digital imaging devices suitable for microscopy and for data analysis also are well known in the art.

A method of modulating neuronal plasticity in an individual in need thereof, said method comprising modulating interaction between Eph receptors on postsynaptic neurons and ephrin ligands on presynaptic neurons. In this case, modulation can be achieved by contacting neurons with the invention compounds. Another embodiment is a method of improving cognition in an individual, said method comprising increasing clustering of Eph receptors at the synaptic site of postsynaptic neurons or by increasing clustering of ephrin ligands at the synaptic site of presynaptic neurons. Modulation can be achieved in this case as well by contacting neurons with the invention compounds.

“Contacting brain neurons” as used herein with respect to improving cognition refers to the process by which a modulator compound is administered to an individual (e.g. a human) such that the compound gains access and contacts neurons involved in cognition. Administration may be by any suitable means, e.g., by oral, sublingual intravenous, subcutaneous, transcutaneous, intramuscular, intracutaneous, intrathecal, epidural, intraoccular, intracranial, inhalation, rectal, vaginal, and the like. The compound to be administered may be formulated with one or more pharmaceutically acceptable carriers, which can take the form of a cream, lotion, tablet, capsule, pellet, dispersible powder, granule, suppository, syrup, elixir, lozenge, injectable solution, sterile aqueous or non-aqueous solution, suspension or emulsion, patch, and the like. The active compound may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

An “effective amount,” refers to a dose sufficient to provide desirable concentrations of the compound in the vicinity of neurons involved in cognition such that LTP is affected. The specific effective dose level for any particular subject will depend upon a variety of factors including the disorder being treated, the severity of the disorder, the activity of the specific polypeptide or compositions used, the route of administration, the rate of clearance of the specific polypeptide or composition, the duration of treatransmembraneent, the drugs used in combination or coincident with the specific polypeptide or composition, the age, body weight, sex, diet and general health of the subject, and like factors well known in the medical arts and sciences. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. Dosage levels typically fall in the range of about 0.001 up to 100 mg/kg/day; with levels in the range of about 0.05 up to 10 mg/kg/day being preferred.

As used herein the term “mental illness” is understood to encompass a broad category of disorders, each of which displays a unique set of symptoms, characterized by abnormalities in cognition, emotion, mood, or social function, which is severe in level or duration. Some of the more common mental illnesses include depression, bipolar disorder, anxiety disorders, phobias, panic disorders, obsessive-compulsive disorders, schizophrenia. A defect in cognition or a “cognitive disability” involves the brain's inability to process, retrieve, store and manipulate information. It is usually manifested in impairments to attention, orientation and memory. Cognitive disability includes deficits in such tasks as problem-solving, judgement, information processing (reading, writing, mathematics) and behavior. Progressive deterioration of cognitive function is referred to as dementia. Dementia is a result of damage to the brain itself and manifests according to the specific brain damage. Dementia may be caused by a number of specific conditions, including Alzheimer's disease, Crutzfeld Jacob disease, head injuries and conditions resulting from exposure to any of a host of chemical, metabolic, and infectious diseases that exert an impact on the brain (e.g., AIDS, depression (Nestler et al., Neuron, 34(1): 13-25 (2002); drug and alcohol addiction (Nestler et al., Am J Addict,10(3):201-17 (2001); persistent pain (Ren et al., J. Orofac. Pain. 13(3):155-63 (1999).

The ability to improve cognition by administering a compound that modulates neuronal plasticity, therefore, provides a therapeutic strategy for increasing cognition capability in a developing individual or in an individual with diminished cognition capacity. In the latter case, diminished capacity may result from disruption of neural network or death or dysfunction of constituent nerve cells achieved by neurodegenerative diseases and disorders, aging, trauma, exposure to harmful chemical or environmental agents, and the like. Thus, potential cognition disorders that may be treated by the methods of the invention include neurodegenerative diseases such as Alzheimer's disease and related disorders, Parkinson's disease, motor neuropathic diseases such as amyotrophic lateral sclerosis, cerebral palsy, multiple sclerosis, Huntington's disease, Crutzfeld Jacob disease, and the like. Also included are drug addiction, alcohol addiction, persistent pain and some types of classical mental illnesses.

The present invention also includes methods of modulating association between an Eph receptor and an ephrin ligand or between an Eph receptor or a PDZ protein (or PDZ domain) by means of genetic therapy. According to this approach an expression vector containing DNA encoding any of these proteins can be administered to CNS neurons involved in plasticity such that uptake of the vector causes and increase in expression of the particular protein. Generally, one increases only one member of the associating pair so that there is an imbalance in the level of expression of one pair versus that of the other member of that pair. Because association between the receptors is density driven to some extent, modulating will result by changing the amount of only one member of an associating pair.

A variety of vectors are known for expressing a protein in neurons. These include, for example, vectors based on herpes simplex viruses (see, e.g., U.S. Pat. Nos. 6,120,773; 5,641,651; 6,383,738; 6,248,320; 5,851,826; and 5,501,579. DNA encoding a particular Eph receptor, ephrin ligand, or PDZ protein which is cloned into the vector for expression may be obtained by methods well known in the art (see e.g., Sambrook et al., Molecular Cloning: A laboratory Manual, Cold Spring Harbor Laboratory Press, 1989). For example, encoding DNA may be prepared synthetically based on published sequences available in scientific journals or in DNA database repositories (see e.g., GenBank). Alternatively, the encoding DNA may be cloned by PCR amplification of genomic DNA or cDNA using primers based on the published sequences.

The invention will be described in greater detail by reference to the following non-limiting examples.

### EXAMPLES

### Example 1

### Postsynaptic Administration of GluR2 C-Terminal Peptide Inhibits Mossy Fiber LTP

The role of the GluR2 subunit in LTP at mossy fiber terminals was evaluated in whole cell patch clamp experiments using hippocampal CA3 pyramidal neurons. Briefly, transverse hippocampal slices (350 μm) were made from P12-18 mice (strain 129SvEv) as previously described (Contractor et al., Neuron 29, 209-16. (2001)). Slices were transferred to a recording chamber, and whole-cell patch clamp recordings made from visually identified pyramidal cells in the CA3 region of the hippocampus. The recording electrode (i.e., patch electrode) is filled with an “internal” solution described below. A seal between the glass of the electrode and the membrane of the cell is made. The seal is then “broken through” by pushing the electrode through the membrane to gain low resistance access to the cell interior and enable voltage clamping of the cell membrane. This also allows dialysis of the cell with test reagents such as peptides and antibodies. A stimulating electrode is attached to a current source and allows current to stimulate action potentials while a reference electrode is placed in the recording bath. The composition of the internal solution was: 95 mM CsF, 25 mM CsCl, 10 mM Cs-HEPES, 10 mM Cs-EGTA, 2 mM NaCl, 2 mM Mg-ATP, 10 mM QX-314, 5 mM TEA-Cl, 5 mM 4-AP, pH adjusted to 7.3 with CsOH.

Peptides were added directly to the internal solution on the day of the experiment from frozen stocks in protease inhibitors made up in phosphate buffered saline (PBS). To avoid leakage of peptides or antibodies into the extracellular space, pipette tips were filled with normal internal solution and then the pipette was backfilled with peptide/antibody containing internal fluid. The final concentration of peptides was 50 μM in all experiments. The final concentration of protease inhibitors were: bestatin 2 μg/ml, leupeptin 25 ng/ml, pepstatin 35 ng/ml, aprotinin 100 ng/ml.

In control experiments where peptides were not included, internal solutions were made up in exactly the same way with protease inhibitors in PBS but lacking any added peptide. Antibodies were made up in PBS stock and added directly to the internal solution at a final concentration of 20 μg/ml. The pH and osmolarity of all internal solutions were checked before use. Series resistances (Rs) were generally <10M and were continuously monitored throughout the duration of the experiment. Recordings in which Rs significantly changed were discarded. Experiments using mutant mice were performed with heterozygous breedings blind to the genotype of the animal.

Mossy fiber EPSCs were evoked with a monopolar glass electrode positioned in the stratum lucidum. EPSCs were evaluated using a 40 ms interval between recordings. All recordings were made in the continuous presence of bicuculline (10 μM), picrotoxin (50 μM) and the NMDA receptor antagonist D-AP5 [D(-)-2-Amino-5-phosphonopentanoic acid; 50 μM]. The group II mGluR agonist, LCCG-1 (10 μM), which selectively depresses mossy fiber transmission, was applied at the end of each experiment. LTP was induced by tetanic stimulation comprising three 1 s stimulations (100 Hz) with a 10 s interval. Data are presented as mean±SEM. Parameters were compared using the Student's unpaired t-test where not specified, and the K-S test, p<0.05 was considered significant.

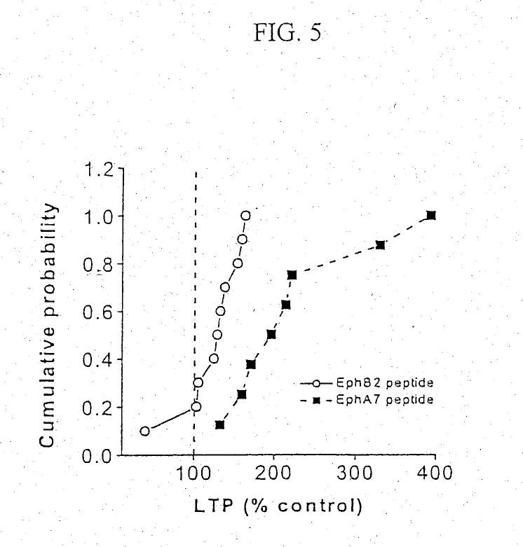

Cumulative probability is determined by ranking the EPSC data set from the highest to the lowest value from 20 to 30 minutes post tetanization. Each point is graphed with its corresponding percentile. For instance in FIG. 5, the EphB2 data taken at 20 to 30 minutes post tentanization shows ten recordings. The lowest LTP value was about 40% signified by the lowest point on the 1 st graph (open circles) and the largest LTP was about 160%.

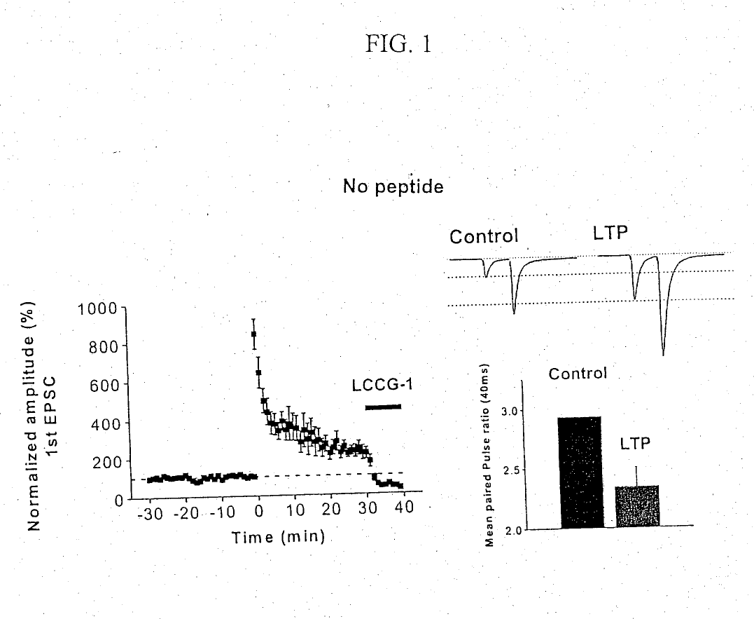

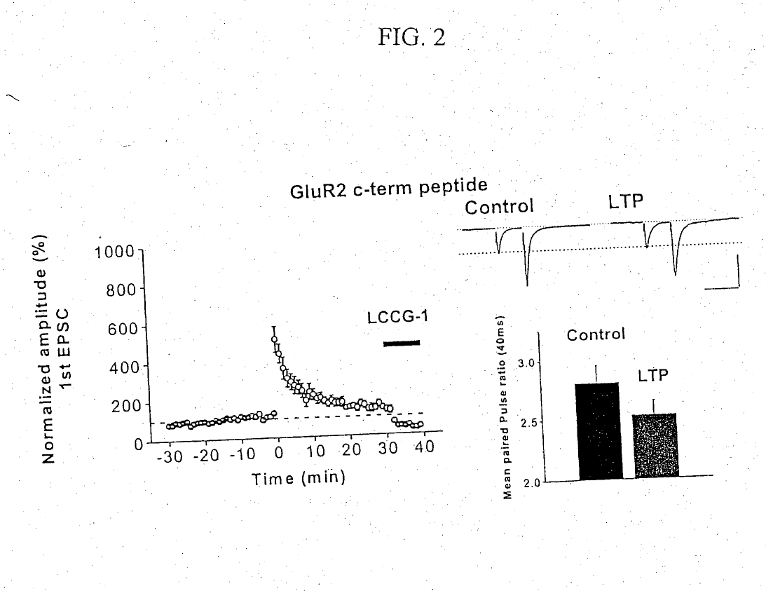

Postsynaptic CA3 pyramidal neurons were perfused intracellularly with a peptide corresponding to the last 10 amino acids of the GluR2 carboxy terminal (R2ct; Asn-Val-Tyr-Gly-Ile-Glu-Ser-Val-Lys-Ile.; SEQ ID NO:1). This peptide, which was previously found to disrupt GluR2-PDZ interactions, achieved a small effect on basal synaptic transmission in some neurons (FIG. 2), not unlike that previously reported for CA1 pyramidal neurons (Daw et al., Neuron 28, 873-86. (2000)). In contrast, potentiation of the excitatory postsynaptic current (EPSC) measured 25 to 30 minutes after tetanic stimulation was significantly smaller with peptide (FIG. 2) than in control recordings without peptide (FIG. 2; control LTP: 220±30%, n=13; GluR2ct LTP: 140±11%, n=18, p<0.01 Kolmogorov-Smirnov (K-S) test; FIGS. 1-3). In control recordings (FIG. 1), the paired-pulse ratio (PPR) of mossy fiber EPSCs, measured at a 40 ms interval between stimuli, was reduced after induction of LTP, consistent with an increase in glutamate release probability previously demonstrated to underlie LTP at mossy fiber synapses (Zalutsky et al. Science 248, 1619-24. (1990)) (FIG. 1; control PPR: 2.9±0.12; LTP PPR: 2.3±0.16, n=12, p<0.01). Postsynaptic perfusion of the GluR2ct peptide reduced this change in PPR (FIG. 2; GluR2ct control PPR: 2.8±0.14; LTP PPR, 2.5±0.12, n=16 p>0.05), in accord with the diminished potentiation after tetanic stimulation. Further, it was observed that short term potentiation measured immediately following tetanic stimulation was significantly smaller in recordings in which the peptide was perfused into the postsynaptic cell (control PTP: 840±70%; GluR2ct PTP: 510±67%, p<0.01). There was no difference in the suppression of mossy fiber EPSCs by the group II mGluR agonist LCCG-1 (p>0.05), which ruled out the possibility that the observed effects were due to significant contamination by non-mossy fiber inputs. See Kamiya et al., J. Physiol. (Lond.) 493, 447-55 (1996). In summary, these initial results clearly indicate a postsynaptic component to induction of short- and long-term potentiation at the mossy fiber synapse.

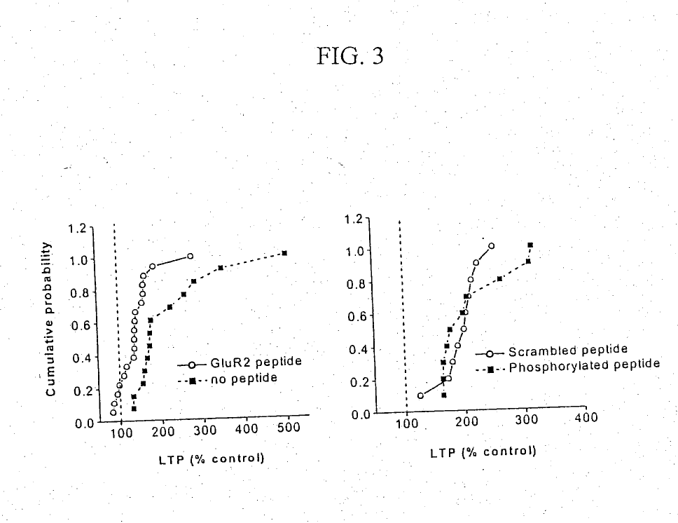

To test the specificity of the GluR2ct peptide effect, recordings were made during intracellular perfusion of a scrambled peptide in which the PDZ recognition consensus sequence has been removed (R2ctSC; Asn-Val-Ile-Tyr-Val-Lys-Ser-Glu-Ile-Gly; SEQ ID NO: 2). Basal EPSC amplitudes were stable in these experiments, LTP was elicited by tetanic stimulation (200±11%, n=10, p>0.05 (K-S test)) (FIG. 3), and PPR was reduced after induction of LTP (control PPR: 3.1±0.19; LTP PPR: 2.4±0.10, n 10, p<0.01). These experiments demonstrate that the LTP inhibitory effect of the GluR2ct peptide is sequence specific and requires the presence of the carboxy terminal PDZ-binding region.

To investigate the type of PDZ protein that was involved in mediating mossy fiber LTP, recordings were made while perfusing CA3 neurons with a phosphorylated GluR2/3 peptide (R2ctPO₄; same as SEQ ID NO: 1 except the serine is phosphorylated), which competes for binding to PICK1, but not GRIP (Matsuda et al., J. Neurochem. 73, 1765-8. (1999); Chung et al., J. Neurosci. 20, 7258-67. (2000)). Tetanic stimulation produced normal potentiation of EPSCs after perfusion of GluR2ctPO4 (210±19%, n=10, p>0.05 (K-S test) compared to control LTP) (FIG. 3) and a reduction in PPR (control PPR: 3.0±0.18; LTP PPR: 2.5±0.16, n=10, p<0.05). Because the phosphorylated peptide did not affect synaptic plasticity, these data indicate that a postsynaptic protein containing a type II PDZ domain such as GRIP is involved in the induction of mossy fiber LTP.

### Example 2

### Role of Eph Receptors and PDZ Protein in Mossy Fiber Synaptic LTP for Pyramidal CA3 Hippocampal Neurons

The observation that postsynaptic perfusion of the GluR2ct peptide inhibited mossy fiber LTP, which is expressed as an increased presynaptic release probability, required consideration of potential mechanisms of retrograde signaling from the postsynaptic neuron to the presynaptic terminal (Fitzsimonds et al. Proc. Physiol. Rev. 78, 143-70. (1998)). Attention was directed to potential trans-synaptic signaling molecules that interact with PDZ domains on GRIP. One such pair of candidate molecules is the Eph receptor tyrosine kinases (EphRs) and their intercellular binding partners, the ephrin ligands. EphR/ephrin intercellular signaling pathways are important for axon guidance and neuronal migration during development (Klein, Curr. Opin. Cell Biol. 13, 196-203. (2001)), but these molecules also are expressed at synapses in adult brain (Torres et al., Neuron 21, 1453-63. (1998); Buchert et al., J. Cell Biol. 144, 361-71. (1999)), including in the CA3 region of the hippocampus (Moreno-Flores et al., Neuroscience 91, 193-201 (1999)), and they interact with the same PDZ proteins as AMPA receptors, albeit at different PDZ domains (Torres et al., Neuron 21, 1453-63. (1998)). Moreover, these receptors interact with NMDA receptors (Dalva et al., Cell 103, 945-56. (2000)) and modulate their function (Takasu et al., Science 295, 491-5. (2002)) and have recently been implicated in NMDA receptor-dependent plasticity (Henderson et al., Neuron 32, 1041-56. (2001); Grunwald et al., Neuron 32, 1027-40. (2001)).

To determine if postsynaptic EphRs have a role in mossy fiber LTP, CA3 pyramidal neurons were perfused with peptides corresponding to the PDZ-binding carboxy terminals of one of the representative members of the two families of Eph receptors, EphB2 (Gln-Met-Asn-Gln-Ile-Gln-Ser-Val-Glu-Val; SEQ ID NO:3) and EphA7 (Leu-His-Leu-His-Gly-Thr-Gly-Ile-Gln-Val; SEQ ID NO: 4).

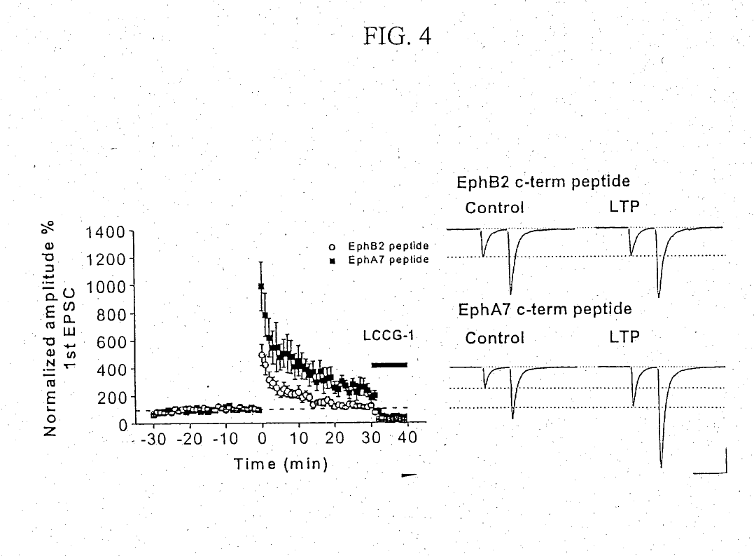

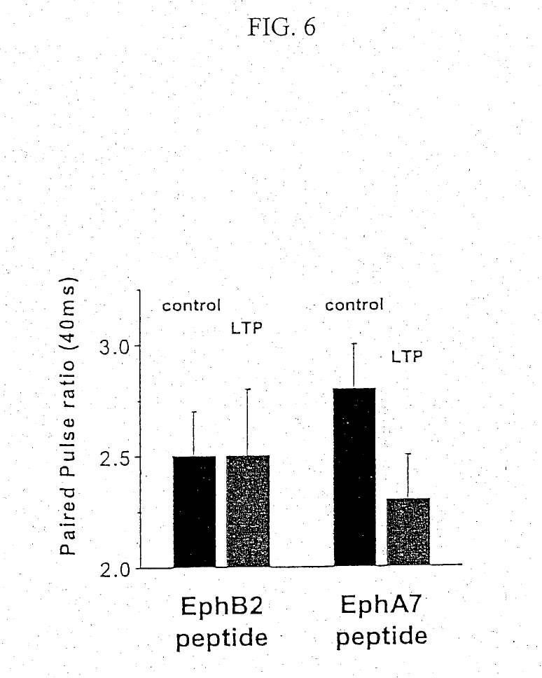

The EphA7 peptide did not affect either short-term plasticity or LTP (control PPR: 2.8±0.15; LTP PPR: 2.3±0.22 p<0.05; PTP: 990±180%; LTP: 250±46%, n=5, p>0.05 (K-S test)) (FIGS. 4-6). In contrast, the EphB2 peptide significantly depressed both post-tetanic potentiation (500±76%, n=7 p<0.05 compared to control recordings) and tetanus induced LTP (125±15%, n=7, p<0.01 (K-S test)) (FIGS. 4-6). In addition, the PPR after tetanic stimulation was identical to that at the end of the basal recording period (control PPR: 2.5±0.22; LTP PPR: 2.5±0.27, n 7, p>0.05). These results support an integral role for interactions between one or more members of the EphB receptor tyrosine kinase family and PDZ-containing proteins such as GRIP in controlling fundamental aspects of mossy fiber release probability.

### Example 3

### Competitive Binding Assay Between GRIP PDZ Domain Fragments and EphB Receptor C-Terminal Fusion Protein

A competitive binding assay was prepared to determine if the LTP inhibitory effects of the GluR2ct peptide were mediated through disruption of binding between Eph receptors and PDZ domain 6 of GRIP (Torres et al., Neuron 21, 1453-63. (1998)) as opposed to disruption of binding between the GluR2 subunit and the GluR2-binding PDZ 4 and 5 domains of GRIP (Dong et al., Nature 386, 279-84. (1997)). For this purpose, a binding assay was established between GRIP PDZ domain fragments (His-GRIP-PDZ4-6) and an EphB2 carboxy terminal fusion protein labeled with ³⁵S-methionine (³⁵S-EphB2).

Briefly, the carboxy terminal region of EphB2 corresponding to mouse EphB2 receptor (amino acid 930-993) was generated by PCR from mouse brain library and subcloned into pGEX2TK (Pharmacia). The GRIP(PDZ4-6) construct, corresponding to GRIP1 (amino acid 448-792) was obtained by PCR from an adult rat hippocampal library and subcloned into pRSET vector. E coli (strain BL21) were transformed with His-GRIP(PDZ4-6) and GST-EphB2 and protein production induced with IPTG. GST-EphB2 protein was purified on glutathionine beads and ³⁵S-EphB2 cleaved from GST with thrombin (1 U/μg). GRIP(PDZ4-6) protein was purified on Ni-NTA agarose, and eluted with imadazole. ³⁵S-EphB2 (50 nM) was incubated with (15 μg) of His-GRIP(PDZ4-6) protein in PBS buffer with protease inhibitors for 2 hrs at 22° C. in the absence or presence of peptides. ³⁵S-EphB2 binding to His-GRIP(PDZ4-6) was determined after a 45 min incubation with Ni-NTA agarose and vacuum filtration onto Whatman GFB filter circles (pretreated with 3% BSA). Filters were washed with ice-cold PBS buffer, allowed to equilibrate overnight in ECOLUME scintillation cocktail (ICN pharmaceuticals, Irvine Calif.), and specific counts measured in a scintillation counter. Non-specific binding was defined as the residual binding measured in the absence of His-GRIP(PDZ4-6).

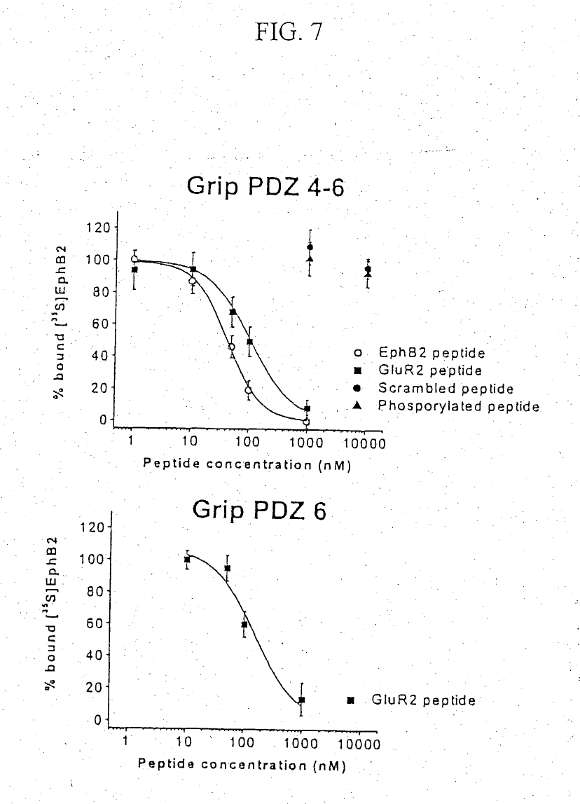

Binding between ³⁵S-EphB2 and His-GRIP-PDZ 4-6 was effectively displaced by the GluR2ct peptide with a Ki of 106 nM (obtained from fitting the data points with the Hill equation) (FIG. 7, top panel). The EphB2 peptide was more potent (Ki value of 43 nM, FIG. 7, top panel). The GluR2ctSC and GluR2ctPO₄ peptides, which did not inhibit LTP, were unable to displace binding between ³⁵S-EphB2 and His-GRIP-PDZ 4-6 in this assay at concentrations as high as 10 μM (FIG. 7, top panel). To test if the GluR2ct peptide displaced EphB2 binding directly from PDZ 6, rather than through an indirect allosteric modulation, the above experiment was repeated using His-GRIP-PDZ 6 in place of His-GRIP-PDZ 4-6. GluR2ct peptide displaced ³⁵S-EphB2 binding to the His-GRIP-PDZ6 fragment with a Ki value of 155 nM (FIG. 7, bottom panel).

### Example 4

### Antibodies Directed Against the Carboxy Terminal of EphB2 as Inhibitors of LTP

A glutathionine-S-transferase (GST) pull down assay was used to ascertain whether EphR and GluR2 are bound to GRIP in the same complex in vivo. In this regard, glutathionine beads containing GST-EphB2 (carboxy terminus), GST alone or GST-NR1a (carboxy terminus) were each incubated with 200 μg of rat brain membranes at 4° C. for 3 hours. The beads were then washed in (4×1 ml) of buffer containing 10 mM Tris, pH 7.4, 0.1 M NaCl, 1 mM EDTA, and 1% Triton. Bound material was eluted from the beads and run on SDS PAGE, transferred to PVDF (polyvinyl-difluoride) membrane and blotted with antibody to GluR2/3.

The pull-down results showed GluR2 specifically associated with EphB2 but not with GST alone or with NMDA receptors (FIG. 8), suggesting that EphB2 and GluR2 can potentially bind to the same GRIP complex in vivo. It was also found that carboxy terminus of full length GluR2, unlike that of the c-terminal peptide, does not bind to PDZ domain 6 of GRIP in yeast as previously described (Dong et al., Nature 386, 279-84. (1997)). Although not wishing to be bound by any theory, the short peptide may have lost its specificity due to a loss in the secondary structure normally needed to restrict the GluR2 carboxy terminus interaction in vivo with PDZ 4 of GRIP. In summary, the results showed that the GluR2ct peptide, unlike the carboxy terminal domain of the GluR2 protein, binds directly to PDZ domain 6 of GRIP and disrupts the interaction between GRIP and EphRs.

### Example 5

### Antibodies Directed Against the Carboxy Terminal of EphB2 as Inhibitors of LTP

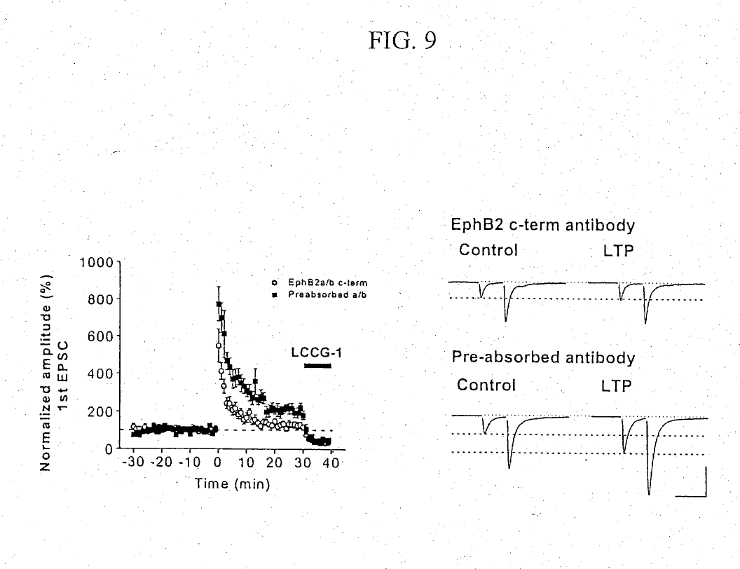

The role played by EphB receptors in LTP was also evaluated by antibody inhibition studies. In this regard, antibody directed against the carboxy terminal of EphB2 (EphB2a/b) was added to intracellular recording solution to disrupt function (FIG. 9). Plasticity after perfusion with the EphB2 antibody was compared to two control conditions in which CA3 pyramidal neurons were either perfused with EphB2 antibody pre-absorbed with a GST fusion protein of the carboxy terminal domain of EphB2 (pre-EphB2a/b) or with an antibody directed against the amino terminus of the EphB1 receptor (EphB1a/b), which is located extracellularly.

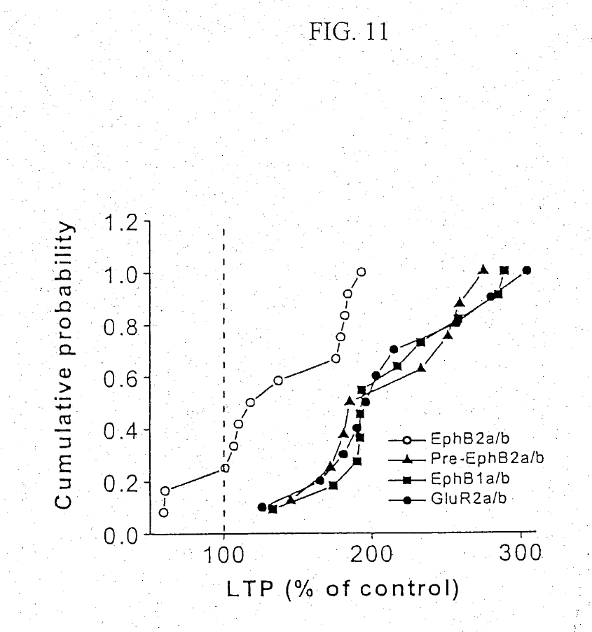

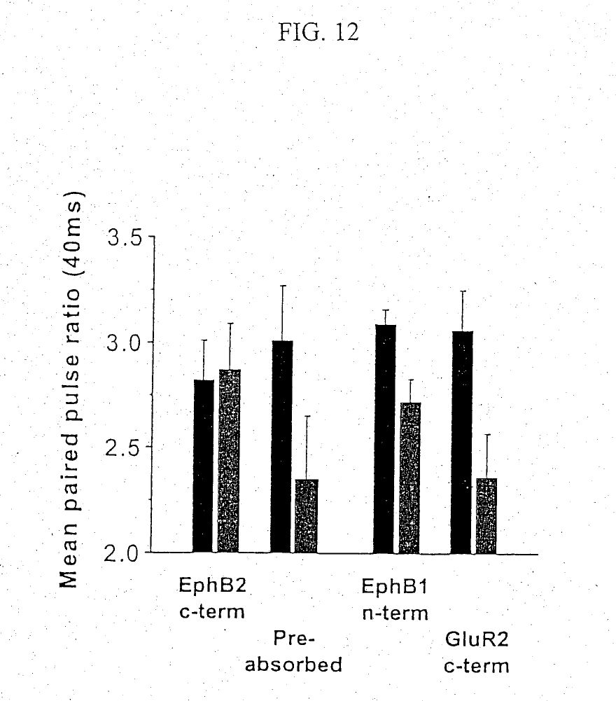

Inclusion of antibody to EphB2a/b had no effect on basal transmission during a 30-minute baseline recording period. However, LTP measured at 20-30 minutes after tetanus was significantly impaired compared to control experiments with the pre-EphB2a/b or EphB1a/b (EphB2a/b LTP: 134±14%, n=12; pre-EphB2a/b LTP: 213±17%, n=8, p<0.05; EphB1a/b LTP: 214±15%, n=11, p<0.05 (K-S test)) (FIGS. 9-11). Similar to previous results, paired-pulse ratios decreased after LTP in the control antibody recordings but did not change significantly when LTP was conducted in the presence of the EphB2 antibody (EphB2a/b control PPR: 2.8±0.2, EphB2a/b LTP PPR: 2.9±0.2, n=12, p>0.05) (FIG. 12). These data provide further support for postsynaptic interactions mossy fiber plasticity that involve carboxy terminal of EphB receptors and intracellular proteins.

### Example 6

### AMPA Receptors are not Involved in LTP of Mossy Fiber Terminals

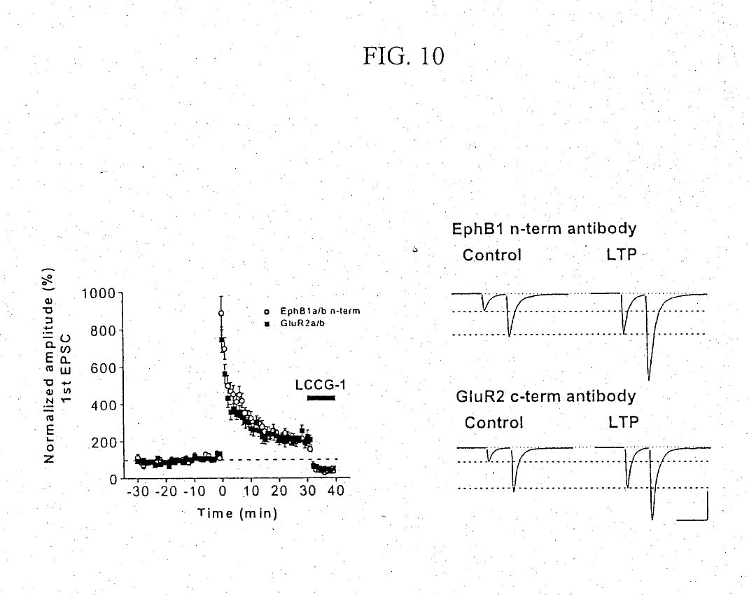

The results in which the GluR2ct peptide inhibited LTP might reflect participation of AMPA receptor PDZ interactions in the induction of mossy fiber LTP. To evaluate this possibility, neurons were perfused with antibody directed against the carboxy-terminal domain of the GluR2 and GluR3 receptor subunits (GluR2a/b). Mossy fiber LTP was normal when this antibody was included in the patch electrode (210±17%, n=10, p>0.05 (K-S test)) (FIG. 10-12). As expected, a concomitant decrease in paired pulse ratio was observed after LTP induction (control PPR: 3.1±0.2; LTP PPR: 2.4 ±0.2, n=10, p<0.05). Thus, the failure of antibodies specific for C-terminus of GluR2 subunits of AMPA receptors to inhibit mossy fiber LTP indicates that AMPA receptors are not involved in LTP induction in these neurons. Thus, the ability of the GluR2/3ct peptide to inhibit mossy fiber LTP occurs through the peptide's non-selective action on the Eph receptor-GRIP interaction.

### Example 7

### EphB2 Receptors and Ligands as Modulators of Transynapytic Signaling Underlying LTP of Mossy Fiber Terminals

To evaluate if trans-synaptic signaling underlies plasticity at the mossy fiber synapse, soluble chimeric protein reagents containing the extracellular domains of the EphB2 receptor (EphB2-Fc;), EphA5 receptor (EphA5-Fc) or ephrin-B1 ligand (ephrin-B1-Fc) were fused to the Fc region of human immunoglobulin (IgG) as described previously (Bruckner et al., Science 275, 1640-3. (1997)). EphB2-Fc and ephrinB1-Fc are available commercially (see, e.g., R&D Systems, Minneapolis, Minn.; Catalog nos. 467-B2, 473EB, respectively). EphB2-Fc from the N-terminus to C-terminus constituted mouse EphB2 (Met 1 to Lys 546), a linker (Asp-Ile-Glu-Gly-Arg-Met-Asp; SEQ ID NO:5), Human gamma heavy chain (Pro 100 to Lys 330) and (His)₅. EphrinB1-Fc from the N-terminus to C-terminus constituted mouse ephrin (Met 1 to Ser 229), a linker (Ile-Glu-Gly-Arg-Met-Asp; SEQ ID NO:5), Human gamma heavy chain (Pro 100 to Lys 330) and (His)₅.

These extracellular domain fusion proteins are used as affinity reagents to recognize their cognate interacting partners expressed on cell surfaces. Because IgG Fc forms a dimer when expressed, the extracellular domains in the fusion proteins were expressed as a dimer. Dimeric forms of the extracellular domains used in these experiments act primarily as a blocking reagent to prevent signaling, and display only weak agonist activity (Stein et al., Genes Dev 12, 667-78. (1998)). In contrast, higher multiples of the Eph-Fc and ephrin-Fc reagents can strongly activate reverse or forward signaling, respectively.

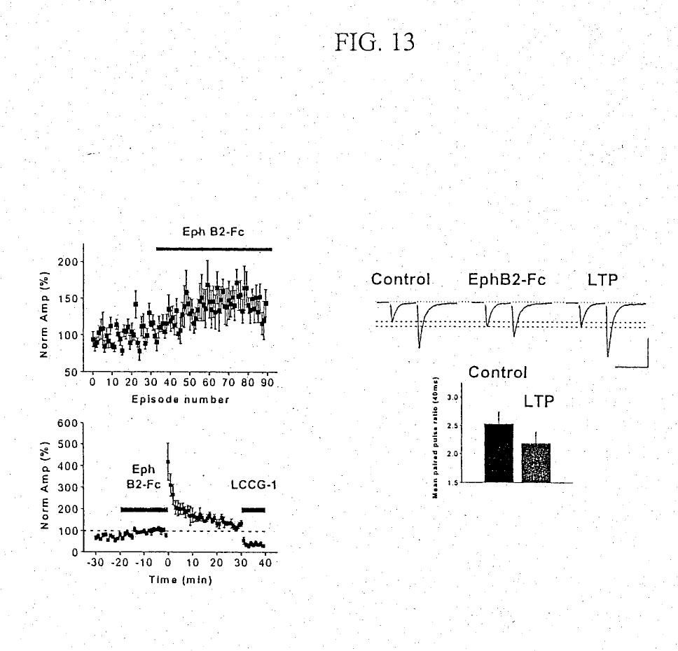

Extracellular bath application of the EphB2-Fc fusion protein (5 μg/ml), which binds to presynaptic ephrins, for a twenty minute period during baseline recording resulted in a 145±13% increase in the basal EPSC amplitude (FIG. 13). In addition, there was a small, non-significant reduction in the baseline PPR. Potentiation of EPSCs after subsequent tetanic stimulation was significantly reduced to a magnitude similar to that observed in the peptide experiments (EphB2-Fc LTP: 134±13%, n=7, p<0.05), and the PPR was not reduced from baseline values (control PPR: 2.5±0.3; LTP PPR: 2.2 ±0.2, n=7, p>0.05) (FIGs. R. Torres et al., Neuron 21, 1453-63. (1998); Moreno-Flores et al. Neuroscience 91, 193-201 (1999).).

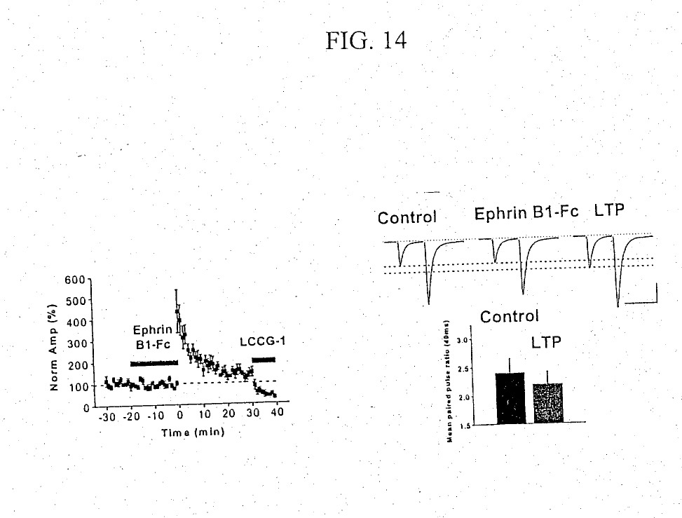

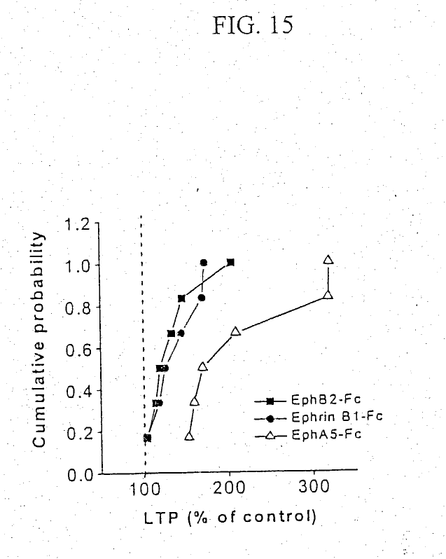

Bath application of the ephrin-B1-Fc fusion protein (5 μg/ml) (which binds postsynaptic Eph receptors), in contrast to EphB2-Fc, did not alter the baseline EPSC amplitudes (FIG. 14). However, LTP after tetanus again was reduced in the presence of ephrinB1-Fc (139±12%, n=6, p<0.05 (K-S test) compared to control LTP) (FIG. 14). Interestingly, post-tetanic potentiation was significantly impaired in the presence of EphB1-Fc (FIGS. 14 and 15), similar to that seen for EphB2-Fc (EphB2-Fc PTP: 420±85%; ephrin-B1-Fc PTP: 430±100%). In contrast, baseline EPSC amplitudes and short- and long-term plasticity were not affected by bath application of the EphA5-Fc (EphA5 LTP: 221±32, n=6, p>0.05 compared to 7 control LTP) (FIG. 15). In summary, these data show that dimeric EphB-Fc fusion proteins can partially activate presynaptic ephrin-B ligands (Stein et al., Genes Dev 12, 667-78. (1998)) and potentiate mossy fiber synaptic transmission, thus occluding subsequent tetanus induced LTP. Soluble ephrin-Fc ligands also block signaling from postsynaptic EphB receptors and significantly impair LTP induction. These data confirm that trans-synaptic interactions between EphB receptors and their ephrin-B ligands mediate a significant component of short- and long-term plasticity at mossy fiber synapses.

### Example 8

### The Effects of PKA Activation Through Forskolin Inhibits Mossy Fiber LTP Downstream of Eph/Ephrin Transynaptic Signaling

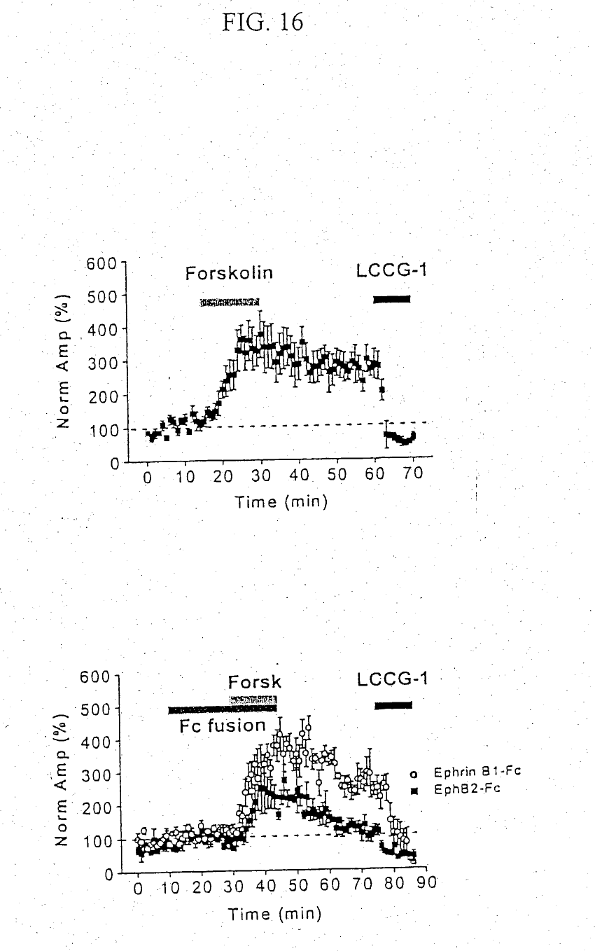

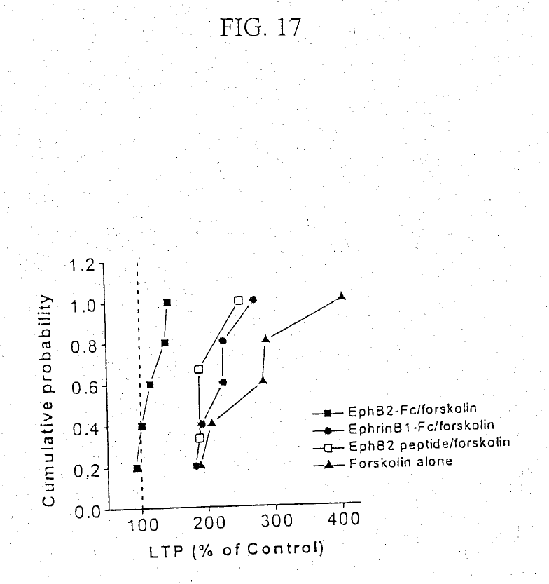

Activation of protein kinase A (“PKA”) by application of forskolin enhances mossy fiber synaptic transmission and inhibits further tetanus-induced LTP. Huang et al., Science 265, 1878-82 (1992). The effect of forskolin-mediated potentiation on Eph/ephrin signaling at mossy fiber synapses was evaluated. In control experiments, bath application of forskolin potentiated mossy fiber EPSCs 20-30 minutes after forskolin application by 275±38%, n=5 (FIGS. 16 and 17; Dalva et al., Cell 103, 945-56. (2000); Takasu et al. Science 295, 491-5. (2002)). Blockade of the interaction between postsynaptic Eph receptors and presynaptic ephrins by pre-application of ephrin-B1-Fc fusion protein did not result in a significant block of potentiation (219±15%, n=5, p>0.05) (FIGS. 16 and 17) indicating that PKA acts downstream of Eph receptor binding to ephrins. When ligands for the presynaptic ephrins (EphB2-Fc) were bath applied, a significant potentiation of baseline amplitudes was again observed (148±19%, n=5). In these experiments, subsequent application of forskolin caused a transient potentiation of mossy fiber EPSCs, however this decayed back to baseline levels 30 mins after washout (118±10%, n=5, p<0.01 compared to control forskolin) (FIGS. 16 and 17). These data demonstrate that activation of presynaptic ephrins inhibits further PKA mediated potentiation of mossy fiber transmission, indicating that ephrins and PKA are part of the same presynaptic signaling pathway that leads to mossy fiber potentiation. This interpretation is further supported by recordings with the EphB2 peptide in the recording electrode. Unlike tetanus-induced LTP, which was blocked by this peptide, forskolin-mediated potentiation was normal in the presence of the EphB2 peptide (209±20%, n=3, p>0.05) (FIG. 17). In summary, these data are consistent with the interpretation that PKA activation is downstream of both GRIP-Eph receptor interaction and trans-synaptic signaling through Eph receptors and ephrin ligands.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety. Definitions that are contained in text incorporated by reference are excluded to the extent that they contradict definitions in this disclosure.

What is claimed is:

1. A method of identifying a compound that modulates neuronal plasticity, said method comprising identifying those candidate compounds which modulate association between an Eph receptor or fragment thereof and an ephrin ligand or fragment thereof, or a PDZ protein or fragment thereof and an Eph receptor or fragment thereof,

2. The method of claim 1, wherein said PDZ protein is GRIP.

3. The method of claim 1, wherein said PDZ protein is a polypeptide that comprises a type II PDZ domain.

4. The method of claim 1, wherein said Eph receptor fragment is a C-terminal fragment.

5. The method of claim 1, wherein said compound comprises a peptide fragment of GluR2 or Eph receptor.

6. The method of claim 1, wherein said modulation is an increase in association.

7. The method of claim 1, wherein said modulation is a decrease in association.

8. The method of claim 1, wherein said Eph receptor is an EphB receptor.

9. A method of screening compounds as potential modulators of neuronal plasticity, said method comprising applying the compounds to postsynaptic neurons of a neuronal synapse and determining whether a biochemical affect is observed at the presynaptic neuron of the synapse.

10. The method of claim 9, wherein said biochemical affect is activation of ephrin-B ligands or activation of protein kinase A.

11. The method of claim 9, wherein said neurons are from hippocampus, cerebellum, cortico-thalamic or amygdala.

12. The method of claim 11, wherein said neurons are hippocampal mossy fiber CA3 neurons.

13. A method of screening compounds as potential modulators of neuronal plasticity, said method comprising applying the compounds to postsynaptic neurons of a neuronal synapse and determining clustering of Eph receptors or presynaptic ephrin ligands.

14. The method of claim 13, wherein said Eph receptor is an Eph B receptor.

15. The method of claim 13, wherein said neurons are from hippocampus, cerebellum, cortico-thalamic or amygdala.

16. The method of claim 13, wherein said neurons are hippocampal mossy fiber CA3 neurons.

17. The method of claim 13, wherein said clustering is mediated by a PDZ protein or fragment thereof.

18. The method of claim 13, wherein said ephrin ligand is an ephrin B ligand.

19. Compounds identified by the method of claim 1.

20. Compounds identified by the method of claim 9.

21. Compounds identified by the method of claim 13.

22. A method of modulating neuronal plasticity in an individual in need thereof, said method comprising contacting neurons of the individual with an effective amount of a compound that modulates interaction between Eph receptors on postsynaptic neurons and ephrin ligands on presynaptic neurons.

23. The method of 22, wherein said modulation increases long-term potentiation and wherein said compound enhances interaction between Eph receptors with ephrin ligands.

24. The method of 22, wherein said modulation decreases long term potentiation and wherein said compound reduces interaction between Eph receptors with ephrin ligands.

25. The method of claim 22, wherein said neurons are from hippocampus, cerebellum, cortico-thalamic or amygdala.

26. The method of claim 22, wherein said neurons are hippocampal mossy fiber CA3 neurons.

27. The method of claim 22, wherein said ephrin ligands are ephrin-B ligands.

28. The method of claim 22, wherein said Eph receptors are Eph B receptors.

29. A method of modulating neuronal plasticity in an individual in need thereof, said method comprising modulating interaction between Eph receptors on postsynaptic neurons and ephrin ligands on presynaptic neurons.

30. The method of 29, wherein said modulation increases long-term potentiation and wherein the interaction between Eph receptors with ephrin ligands is enhanced.

31. The method of 29, wherein said modulation decreases long term potentiation and wherein the interaction between Eph receptors with ephrin ligands is reduced.

32. The method of claim 29, wherein said neurons are from hippocampus, cerebellum, cortico-thalamic or amygdala.

33. The method of claim 29, wherein said neurons are hippocampal mossy fiber CA3 neurons.

34. The method of claim 29, wherein said ephrin ligands are ephrin-B ligands.

35. The method of claim 29, wherein said Eph receptors are Eph B receptors.

36. A method of improving cognition in an individual, said method comprising contacting brain neurons of the individual with an effective amount of a compound that increases clustering of Eph receptors at the synaptic site of postsynaptic neurons or increases clustering of ephrin ligands at the synaptic site of presynaptic neurons.

37. The method of claim 36, wherein said neurons are from hippocampus, cerebellum, cortico-thalamic or amygdala.

38. The method of claim 36, wherein said neurons are hippocampal mossy fiber CA3 neurons.

39. The method of claim 36, wherein said Eph receptors are Eph B receptors.

40. A method of improving cognition in an individual, said method comprising increasing clustering of Eph receptors at the synaptic site of postsynaptic neurons or by increasing clustering of ephrin ligands at the synaptic site of presynaptic neurons.

41. The method of claim 40, wherein said neurons are from hippocampus, cerebellum, cortico-thalamic or amygdala.

42. The method of claim 40, wherein said neurons are hippocampal mossy fiber CA3 neurons.

43. The method of claim 40, wherein said Eph receptors are Eph B receptors.
